SEQUENCE LISTING (24 sequences)

SEQ ID NO: 1 - protein, 376 aa, Artificial Sequence
  Synthetic
KSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 2 - protein, 238 aa, Artificial Sequence
  Synthetic
VSATLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 3 - protein, 509 aa, Artificial Sequence
  Synthetic
KEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNL

SEQ ID NO: 4 - protein, 1090 aa, Artificial Sequence
  Synthetic
TSRREVCDCNGKSRQCIFDRELHRQTGNGFRCLNCNDNTDGIHCEKCKNGFYRHRERDRCLPCNCNSKGSLSARCDNSGRCSCKPGVTGARCDRCLPGFHMLTDAGCTQDQRLLDSKCDCDPAGIAGPCDAGRCVCKPAVTGERCDRCRSGYYNLDGGNPEGCTQCFCYGHSASCRSSAEYSVHKITSTFHQDVDGWKAVQRNGSPAKLQWSQRHQDVFSSAQRLDPVYFVAPAKFLGNQQVSYGQSLSFDYRVDRGGRHPSAHDVILEGAGLRITAPLMPLGKTLPCGLTKTYTFRLNEHPSNNWSPQLSYFEYRRLLRNLTALRIRATYGEYSTGYIDNVTLISARPVSGAPAPWVEQCICPVGYKGQFCQDCASGYKRDSARLGPFGTCIPCNCQGGGACDPDTGDCYSGDENPDIECADCPIGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPADKCRACNCNPMGSEPVGCRSDGTCVCKPGFGGPNCEHGAFSCPACYNQVKIQMDQFMQQLQRMEALISKAQGGDGVVPDTELEGRMQQAEQALQDILRDAQISEGASRSLGLQLAKVRSQENSYQSRLDDLKMTVERVRALGSQYQNRVRDTHRLITQMQLSLAESEASLGNTNIPASDHYVGPNGFKSLAQEATRLAESHVESASNMEQLTRETEDYSKQALSLVRKALHEGVGSGSGSPDGAVVQGLVEKLEKTKSLAQQLTREATQAEIEADRSYQHSLRLLDSVSRLQGVSDQSFQVEEAKRIKQKADSLSSLVTRHMDEFKRTQKNLGNWKEEAQQLLQNGKSGREKSDQLLSRANLAKSRAQEALSMGNATFYEVESILKNLREFDLQVDNRKAEAEEAMKRLSYISQKVSDASDKTQQAERALGSAAADAQRAKNGAGEALEISSEIEQEIGSLNLEANVTADGALAMEKGLASLKSEMREVEGELERKELEFDTNMDAVQMVITEAQKVDTRAKNAGVTIQDTLNTLDGLLHLMGM

SEQ ID NO: 5 - protein, 305 aa, Artificial Sequence
  Synthetic
QPLGGESICSARAPAKYSITFTGKWSQTAFPKQYPLFRPPAQWSSLLGAAHSSDYSMWRKNQYVSNGLRDFAERGEAWALMKEIEAAGEALQSVHEVFSAPAVPSGTGQTSAELEVQRRHSLVSFVVRIVPSPDWFVGVDSLDLCDGDRWREQAALDLYPYDAGTDSGFTFSSPNFATIPQDTVTEITSSSPSHPANSFYYPRLKALPPIARVTLLRLRQSPRAFIPPAPVLPSRDNEIVDSASVPETPLDCEVSLWSSWGLCGGHCGRLGTKSRTRYVRVQPANNGSPCPELEEEAECVPDNCV

SEQ ID NO: 6 - protein, 129 aa, Artificial Sequence
  Synthetic
EEGARLLASKSLLNRYAVEGRDLTLQYNIYNVGSSAALDVELSDDSFPPEDFGIVSGMLNVKWDRIAPASNVSHTVVLRPLKAGYFNFTSATITYLAQEDGPVVIGSTSAPGQGGILAQREFDRRFSPH

SEQ ID NO: 7 - protein, 118 aa, Artificial Sequence
  Synthetic
LRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 8 - protein, 441 aa, Artificial Sequence
  Synthetic
DRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISDVFQQVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 9 - protein, 517 aa, Artificial Sequence
  Synthetic
YHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSG

SEQ ID NO: 10 - protein, 576 aa, Artificial Sequence
  Synthetic
TRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 11 - protein, 145 aa, Artificial Sequence
  Synthetic
APKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPG

SEQ ID NO: 12 - protein, 141 aa, Artificial Sequence
  Synthetic
AAGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSS

SEQ ID NO: 13 - protein, 184 aa, Artificial Sequence
  Synthetic
AGPSSGSCPPTKFQCRTSGLCVPLTWRCDRDLDCSDGSDEEECRIEPCTQKGQCPPPPGLPCPCTGVSDCSGGTDKKLRNCSRLACLAGELRCTLSDDCIPLTWRCDGHPDCPDSSDELGCGTNEILPEGDATTMGPPVTLESVTSLRNATTMGPPVTLESVPSVGNATSSSAGDQSGSPTAYG

SEQ ID NO: 14 - protein, 630 aa, Artificial Sequence
  Synthetic
EPCRAVFREAEVTLEAGGAEQEPGQALGKVFMGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQMTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKGG

SEQ ID NO: 15 - protein, 630 aa, Artificial Sequence
  Synthetic
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 16 - protein, 289 aa, Artificial Sequence
  Synthetic
EVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 17 - protein, 424 aa, Artificial Sequence
  Synthetic
HPDRIIFPNHACEDPPAVLLEVQGTLQRPLVRDSRTSPANCTWLILGSKEQTVTIRFQKLHLACGSERLTLRSPLQPLISLCEAPPSPLQLPGGNVTITYSYAGARAPMGQGFLLSYSQDWLMCLQEEFQCLNHRCVSAVQRCDGVDACGDGSDEAGCSSDPFPGLTPRPVPSLPCNVTLEDFYGVFSSPGYTHLASVSHPQSCHWLLDPHDGRRLAVRFTALDLGFGDAVHVYDGPGPPESSRLLRSLTHFSNGKAVTVETLSGQAVVSYHTVAWSNGRGFNATYHVRGYCLPWDRPCGLGSGLGAGEGLGERCYSEAQRCDGSWDCADGTDEEDCPGCPPGHFPCGAAGTSGATACYLPADRCNYQTFCADGADERRCRHCQPGNFRCRDEKCVYETWVCDGQPDCADGSDEWDCSYVLPRK

SEQ ID NO: 18 - protein, 171 aa, Artificial Sequence
  Synthetic
ADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSKV

SEQ ID NO: 19 - protein, 758 aa, Artificial Sequence
  Synthetic
QESCSMRCGALDGPCSCHPTCSGLGTCCLDFRDFCLEILPYSGSMMGGKDFVVRHFKMSSPTDASVICRFKDSIQTLGHVDSSGQVHCVSPLLYESGRIPFTVSLDNGHSFPRAGTWLAVHPNKVSMMEKSELVNETRWQYYGTANTSGNLSLTWHVKSLPTQTITIELWGYEETGMPYSQEWTAKWSYLYPLATHIPNSGSFTFTPKPAPPSYQRWRVGALRIIDSKNYAGQKDVQALWTNDHALAWHLSDDFREDPVAWARTQCQAWEELEDQLPNFLEELPDCPCTLTQARADSGRFFTDYGCDMEQGSVCTYHPGAVHCVRSVQASLRYGSGQQCCYTADGTQLLTADSSGGSTPDRGHDWGAPPFRTPPRVPSMSHWLYDVLSFYYCCLWAPDCPRYMQRRPSNDCRNYRPPRLASAFGDPHFVTFDGTNFTFNGRGEYVLLEAALTDLRVQARAQPGTMSNGTETRGTGLTAVAVQEGNSDVVEVRLANRTGGLEVLLNQEVLSFTEQSWMDLKGMFLSVAAGDRVSIMLASGAGLEVSVQGPFLSVSVLLPEKFLTHTHGLLGTLNNDPTDDFTLHSGRVLPPGTSPQELFLFGANWTVHNASSLLTYDSWFLVHNFLYQPKHDPTFEPLFPSETTLNPSLAQEAAKLCGDDHFCNFDVAATGSLSTGTATRVAHQLHQRRMQSLQPVVSCGWLAPPPNGQKEGNRYLAGSTIYFHCDNGYSLAGAETSTCQADGTWSSPTPKCQPGRSYA

SEQ ID NO: 20 - protein, 121 aa, Artificial Sequence
  Synthetic
WSPQEEDRIIEGGIYDADLNDERVQRALHFVISEYNKATEDEYYRRLLRVLRAREQIVGGVNYFFDIEVGRTICTKSQPNLDTCAFHEQPELQKKQLCSFQIYEVPWEDRMSLVNSRCQEA

SEQ ID NO: 21 - DNA, 29 bp, Artificial Sequence
  Synthetic
ccggatatcagcaagcccacgtgcccacc

SEQ ID NO: 22 - DNA, 40 bp, Artificial Sequence
  Synthetic
aaggaaaaaagcggccgctcatttacccggagagcgggag

SEQ ID NO: 23 - DNA, 34 bp, Artificial Sequence
  Synthetic
cccaagcttgcagcacccaagtgtgcaccggcac

SEQ ID NO: 24 - DNA, 37 bp, Artificial Sequence
  Synthetic
gtgctcgagtcacgtcagagggctggctctctgctcg

(19) United States
(12) Patent Application Publication
(10) Pub. No.: US 2018/0275129 A1
(43) Pub. Date: Sep. 27, 2018

(54) Reagents and Methods for Breast Cancer Detection

(71) Applicant: Sanford Health, Sioux Falls, SD (US)

(72) Inventors: Kristi EGLAND, Sioux Falls, SD (US); Rick EVANS, Sioux Falls, SD (US); James POTTALA, Sioux Falls, SD (US)

(21) Appl. No.: 16/002,493

(22) Filed: Jun. 7, 2018

Related U.S. Application Data
(63) Continuation-in-part of application No. 14/660,423, filed on Mar. 17, 2015, now Pat. No. 10,001,484.
(60) Provisional application No. 61/954,914, filed on Mar. 18, 2014.

(51) Int. Cl. G01N 33/574

(52) U.S. Cl. CPC G01N 33/57415; G01N 2333/475; G01N 2333/435; G01N 2333/705; G01N 2333/70503; G01N 2333/70596; G01N 2333/912; G01N 2333/71

(57) ABSTRACT

The present invention provides compositions including reagents for detecting human autoantibodies against at least two proteins selected from the group consisting of ANGTPL4, DKK1, EPHA2, LAMC2, SPON2, SSR2, GAL1, GFRA1, LRRC15, CD147, CD320, CDH3, LRP10, SPINT2, SUSD2, and CST2, and their use in detecting breast cancer or disease recurrence.

### CROSS REFERENCE

This application is a continuation of U.S. patent application Ser. No. 14/660,423 filed Mar. 17, 2015, which claims priority to U.S. Provisional Patent Application Ser. No. 61/954,914 filed Mar. 18, 2014, incorporated by reference herein in its entirety.

### BACKGROUND

For patients with breast cancer (BCa), early and personalized diagnosis is crucial for optimizing treatments leading to long-term survival. Although mammography is the most widely used method to detect BCa, approximately 20% of screening mammograms result in a false negative diagnosis largely due to high breast density. Additionally, 1 in 10 women who get a mammogram will need additional imaging. Yet, the overwhelming majority of these women will not have BCa, as only 2 to 4 of every 1,000 screening mammograms leads to a cancer diagnosis. Therefore, there is an urgent clinical need to develop a novel, minimally invasive diagnostic strategy for the early diagnosis and monitoring of BCa.

### SUMMARY OF THE INVENTION

In a first aspect, the invention provides compositions consisting of between 2 and 25 antibody detection markers, wherein the composition includes reagents for detecting human autoantibodies against at least two proteins selected from the group consisting of human ANGTPL4, DKK1, EPHA2, LAMC2, SPON2, SSR2, GAL1, GFRA1, LRRC15, CD147, CD320, CDH3, LRP10, SPINT2, SUSD2, and CST2. In one embodiment, the composition includes reagents for detecting human autoantibodies against at least two proteins selected from the group consisting of human ANGPTL4, DKK1, EPHA2, GAL1, LAMC2, SPON2, CST2, SPINT2 and SSR2. In a further embodiment, the composition includes reagents for detecting human autoantibodies against at least 5 proteins in the recited group. In another embodiment, the composition further includes reagents for detecting human autoantibodies against one or both of MUC1 and GRN. In various embodiments, the composition consists of between 2 and 20, 4 and 10, and 5-10 antibody detection markers. In various further embodiments, the composition includes reagents for detecting human autoantibodies against one of the following marker sets:

### ANGPTL4, DKK1, GAL1, GFRA1, GRANULIN, LRRC15, and MUC1;

### ANGPTL4, DKK1, GAL1, GRANULIN, LRRC15, and MUC1;

### ANGPTL4, DKK1, GAL1, and LRRC15;

### ANGPTL4, DKK1, GAL1, GFRA1, and LRRC15;

### DKK1, GAL1, GFRA1, GRANULIN, LRRC1, and 5 MUC1;

### ANGPTL4, DKK1, GAL1, GFRA1, GRANULIN, and LRRC15;

### DKK1, GAL1, GRANULIN, LRRC15, and MUC1;

### DKK1, GAL1, GFRA1, GRANULIN, and LRRC15;

### DKK1, GAL1, GFRA1, LRRC15, and MUC1;

### ANGPTL4, DKK1, GAL1, GRANULIN, and LRRC15;

### DKK1, GAL1, GFRA1, and LRRC15;

### DKK1, GAL1, GRANULIN, and LRRC15;

### ANGPTL4, DKK1, GAL1, LRRC15, and MUC1;

### DKK1, GAL1, and LRRC15;

### ANGPTL4, GAL1, LRRC15, and MUC1;

### GAL1, GFRA1, LRRC15, and MUC1;

### GAL1, GFRA1, and LRRC15;

### ANGPTL4, GAL1, and LRRC15;

### DKK1, GAL1, LRRC15, and MUC1;

### ANGPTL4, GAL1, GFRA1, and LRRC15;

### GAL1, LRRC15, and MUC1;

### ANGPTL4, GAL1, GFRA1, LRRC15, and MUC1;

### ANGPTL4, GAL1, and GFRA1;

### DKK1, GAL1, and GFRA1; and

### GAL1, GFRA1, and MUC1.

In another embodiment, the reagents for detecting human autoantibodies comprise the at least two proteins, or antigenic fragments thereof. In a further embodiment, the at least two proteins, or antigenic fragments thereof comprise native extracellular domains and/or native secreted proteins or antigenic fragments thereof. In a still further embodiment, the reagents are detectably labeled. In another embodiment, reagents are immobilized on a surface.

In another aspect, the invention provides methods for detecting breast cancer or disease recurrence, comprising contacting a bodily fluid sample from a subject at risk of having breast cancer or breast cancer recurrence with one or more reagents for detecting autoantibodies against one or more of human ANGTPL4, DKK1, EPHA2, LAMC2, SPON2, SSR2, GAL1, GFRA1, LRRC15, CD147, CD320, CDH3, LRP10, SPINT2, SUSD2, and CST2, wherein the presence of autoantibodies against the one or more proteins correlates with a likelihood of the subject having breast cancer or breast cancer recurrence. In another embodiment, the reagents comprise reagents for detecting autoantibodies against one or more of human ANGPTL4, DKK1, EPHA2, GAL1, LAMC2, SPON2, CST2, SPINT2 and SSR2. In various further embodiments, the reagents comprise reagents for detecting autoantibodies two or more, or five or more of the recited proteins. In another embodiment the reagents comprise reagents for detecting human autoantibodies against one of the following marker sets:

### ANGPTL4, DKK1, GAL1, GFRA1, GRANULIN, LRRC15, and MUC1;

### ANGPTL4, DKK1, GAL1, GRANULIN, LRRC15, and MUC1;

### ANGPTL4, DKK1, GAL1, and LRRC15;

### ANGPTL4, DKK1, GAL1, GFRA1, and LRRC15;

### DKK1, GAL1, GFRA1, GRANULIN, LRRC1, and 5 MUC1;

### ANGPTL4, DKK1, GAL1, GFRA1, GRANULIN, and LRRC15;

### DKK1, GAL1, GRANULIN, LRRC15, and MUC1;

### DKK1, GAL1, GFRA1, GRANULIN, and LRRC15;

### DKK1, GAL1, GFRA1, LRRC15, and MUC1;

### ANGPTL4, DKK1, GAL1, GRANULIN, and LRRC15;

### DKK1, GAL1, GFRA1, and LRRC15;

### DKK1, GAL1, GRANULIN, and LRRC15;

### ANGPTL4, DKK1, GAL1, LRRC15, and MUC1;

### DKK1, GAL1, and LRRC15;

### ANGPTL4, GAL1, LRRC15, and MUC1;

### GAL1, GFRA1, LRRC15, and MUC1;

### GAL1, GFRA1, and LRRC15;

### ANGPTL4, GAL1, and LRRC15;

### DKK1, GAL1, LRRC15, and MUC1;

### ANGPTL4, GAL1, GFRA1, and LRRC15;

### GAL1, LRRC15, and MUC1;

### ANGPTL4, GAL1, GFRA1, LRRC15, and MUC1;

### ANGPTL4, GAL1, and GFRA1;

### DKK1, GAL1, and GFRA1; and

### GAL1, GFRA1, and MUC1.

In a further embodiment, the reagents comprise reagents for detecting human autoantibodies against human ANGPTL4, DKK1, GAL1, MUC1, GFRA1, GRN and LRRC15. In another embodiment, the one or more reagents comprise the composition of any embodiment or combination of embodiments of the invention. In a further embodiment, the contacting comprises use of ELISA. In another embodiment, the bodily fluid sample comprises a serum sample from the subject. In a further embodiment, the method identifies the subject as likely to have breast cancer or breast cancer recurrence. In a further embodiment, the method further comprises treating the subject with an amount of a therapeutic sufficient to treat the breast cancer or breast cancer recurrence.

In a further aspect, the invention provides methods for treating a subject with breast cancer, comprising:

(a) testing a bodily fluid sample from a subject at risk of breast cancer, and identifying candidate subjects that:
(i) have autoantibodies against at least one of ANGPTL4, DKK1, GAL1, MUC1, GFRA1, GRN and LRRC15; and/or
(ii) do not have autoantibodies against GFRA1, GRN and/or LRRC15; and

(b) treating the candidate subjects with an amount of a therapeutic sufficient to treat the breast cancer.

In one embodiment, the contacting comprises use of Longitudinal Assay Screening, wherein all target biomarkers may be detected and quantitated within a single test and dilution. In a further embodiment, the bodily fluid sample comprises a blood sample from the subject.

### BRIEF DESCRIPTION OF THE FIGURES

FIG. 1. Antigen conformation affects antibody recognition. A, ELISA analysis using an antigen designed to have native conformation. Wells were coated with anti-rabbit IgG followed by the HER-2-ECD-rFc protein generated in 293T cells. Serial dilutions of anti-HER-2 monoclonal antibodies generated against native HER-2, 3F32 (blue), Herceptin (green) or against denatured HER-2, 3F27 (red) were used in ELISA. Reactions were developed after addition of the appropriate secondary antibody. The O.D. is the absorbance reading for the reaction. B, ELISA analysis using a denatured antigen. Wells were coated with purified His-HER-2-ECD generated in E. coli, and serial dilutions of 3F32 (blue), Herceptin (green) or 3F27 (red) were added. After addition of the secondary antibody, the reactions were developed. C, detection of native HER-2 on SKBR3 cells via flow cytometry. Fluorescence indicates antibody recognition of HER-2 on the surface of SKBR3 cells. D, binding competition assay to demonstrate specificity of conformation-carrying antigen ELISA. Wells were precoated with anti-rabbit IgG followed by HER-2-ECD-rFc. Purified HER-2-Fc (black) or CD30-Fc (purple) chimeric proteins were serially diluted and added to a constant amount of Herceptin before addition to the wells. The reactions were developed after incubation with the secondary antibody.

FIG. 2. ROC curve comparison for classification of breast cancer patients. The autoantibody responses against seven antigens (i.e. ANGPTL4, DKK1, GAL1, GFRA1, GRN, LRRC15 and MUC1) were added to a logistic regression model that included age, BMI, race and current smoking status. The ROC curves were determined for all subjects (top) and by specific subtypes of breast cancer including ER positive, invasive, maximum tumor dimension >1 cm, in situ, lymph node involvement and HER-2 amplification (bottom).

### DETAILED DESCRIPTION OF THE INVENTION

All references cited are herein incorporated by reference in their entirety.

Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

In a first aspect, the present invention provides compositions consisting of between 2 and 25 antibody detection markers, wherein the composition includes reagents for detecting human autoantibodies against at least two proteins selected from the group consisting of human ANGTPL4, DKK1, EPHA2, LAMC2, SPON2, SSR2, GAL1, GFRA1, LRRC15, CD147, CD320, CDH3, LRP10, SPINT2, SUSD2, and CST2. The inventors have unexpectedly discovered that autoantibodies against the recited proteins provide an indication of whether a subject is suffering from breast cancer (BCa). Thus, the compositions of the invention can be used, for example, in diagnostic assays to discriminate between BCa and healthy patients by the detection of antibodies in a sample from the subject or to detect recurrence of disease in a breast cancer patient after treatment. In one embodiment, the composition includes reagents for detecting human autoantibodies against at least two proteins selected from the group consisting of ANGPTL4, DKK1, EPHA2, GAL1, LAMC2, SPON2, CST2, SPINT2 and SSR2.

In various embodiments, the composition includes reagents for detecting human autoantibodies against at least three, four, five, six, seven, eight, nine, ten, eleven, twelve, thirteen, fourteen, fifteen, or sixteen proteins in the recited group. In various further embodiments, the composition consists of between 2-24, 2-23, 2-22, 2-21, 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-25, 3-24, 3-23, 3-22, 3-21, 3-20, 3-19, 3-18, 3-17, 3-16, 3-15, 3-14, 3-13, 3-12, 3-11, 3-10, 3-9, 3-8, 3-7, 3-6, 3-5, 3-4, 4-25, 4-24, 4-23, 4-22, 4-21, 4-20, 4-19, 4-18, 4-17, 4-16, 4-15, 4-14, 4-13, 4-14, 4-13, 4-12, 4-11, 4-10, 4-9, 4-8, 4-7, 4-6, 4-5, 5-25, 5-24, 5-23, 5-22, 5-21, 5-20, 5-19, 5-18, 5-17, 5-16, 5-15, 5-14, 5-13, 5-12, 5-11, 5-10, 5-9, 5-8, 5-7, 5-6, 6-25, 6-24, 6-23, 6-22, 6-21, 6-20, 6-19, 6-18, 6-17, 6-16, 6-15, 6-14, 6-13, 6-12, 6-11, 6-10, 6-9, 6-8, 6-7, 7-25, 7-24, 7-23, 7-22, 7-21, 7-20, 7-19, 7-18, 7-17, 7-16, 7-15, 7-14, 7-13, 7-12, 7-11, 7-10, 7-9, 7-8, 8-25, 8-24, 8-23, 8-22, 8-21, 8-20, 8-19, 8-18, 8-17, 8-16, 8-15, 8-14, 8-13, 8-12, 8-11, 8-10, 8-9, 9-25, 9-24, 9-23, 9-22, 9-21, 9-20, 9-19, 9-18, 9-17, 9-16, 9-15, 9-14, 9-13, 9-12, 9-11, 9-10, 1, 2, 3, 4, 5, 6, 7, 8, 9, 10-11, 12, 13, 14, 15, or 16 antibody detection reagents.

As will be understood by those of skill in the art, the compositions may include additional antibody detection markers and controls as is appropriate for an intended use of the composition. In one non-limiting embodiment, the compositions may further comprise reagents for detecting antibodies against one or both of mucin-1(MUC1), HER-2 (41), IGFBP2, and GRANULIN (GRN).

In further embodiments, the compositions comprise or consist of reagents for detecting human autoantibodies against one of the following marker sets, which are shown in the examples that follow (see Table 5) to provide strong predictive value for diagnosing breast cancer:

### ANGPTL4, DKK1, GAL1, GFRA1, GRANULIN, LRRC15, and MUC1;

### ANGPTL4, DKK1, GAL1, GRANULIN, LRRC15, and MUC1;

### ANGPTL4, DKK1, GAL1, and LRRC15;

### ANGPTL4, DKK1, GAL1, GFRA1, and LRRC15;

### DKK1, GAL1, GFRA1, GRANULIN, LRRC1, and 5 MUC1;

### ANGPTL4, DKK1, GAL1, GFRA1, GRANULIN, and LRRC15;

### DKK1, GAL1, GRANULIN, LRRC15, and MUC1;

### DKK1, GAL1, GFRA1, GRANULIN, and LRRC15;

### DKK1, GAL1, GFRA1, LRRC15, and MUC1;

### ANGPTL4, DKK1, GAL1, GRANULIN, and LRRC15;

### DKK1, GAL1, GFRA1, and LRRC15;

### DKK1, GAL1, GRANULIN, and LRRC15;

### ANGPTL4, DKK1, GAL1, LRRC15, and MUC1;

### DKK1, GAL1, and LRRC15;

### ANGPTL4, GAL1, LRRC15, and MUC1;

### GAL1, GFRA1, LRRC15, and MUC1;

### GAL1, GFRA1, and LRRC15;

### ANGPTL4, GAL1, and LRRC15;

### DKK1, GAL1, LRRC15, and MUC1;

### ANGPTL4, GAL1, GFRA1, and LRRC15;

### GAL1, LRRC15, and MUC1;

### ANGPTL4, GAL1, GFRA1, LRRC15, and MUC1;

### ANGPTL4, GAL1, and GFRA1;

### DKK1, GAL1, and GFRA1; and

### GAL1, GFRA1, and MUC1.

In another embodiment, the compositions comprise or consist of reagents for detecting human autoantibodies against human ANGPTL4, DKK1, GAL1, MUC1, GFRA1, GRN and LRRC15.

The antibody detection markers may be any suitable reagents that can be used to detect antibodies against the recited proteins, including but not limited to the recited protein, a secreted version of the protein (such as a native secreted form of the protein), or an extracellular domain of the protein. Secreted proteins are more easily delivered from tumor cells to lymph nodes, where interactions of immune cells take place resulting in abundant high-affinity antibodies. Membrane surface proteins are commonly released in a soluble form from tumor cells through metalloproteinase-dependent cleavage. The shed proteins are more easily transferred to the lymph nodes than intracellular protein. Thus, in one embodiment the antibody detection marker is a secreted or membrane portion of the recited protein. Exemplary amino acid sequences of the secreted or membrane portion of the recited human proteins are shown below.

ANGTPL4
(SEQ ID NO: 1)
KSPRFASWDEMNVLAHGLLQLGQGLREHAERTRSQLSALERRLSACGSAC
QGTEGSTDLPLAPESRVDPEVLHSLQTQLKAQNSRIQQLFHKVAQQQRHL
EKQHLRIQHLQSQFGLLDHKHLDHEVAKPARRKRLPEMAQPVDPAHNVSR
LHRLPRDCQELFQVGERQSGLFEIQPQGSPPFLVNCKMTSDGGWTVIQRR
HDGSVDFNRPWEAYKAGFGDPHGEFWLGLEKVHSITGDRNSRLAVQLRDW
DGNAELLQFSVHLGGEDTAYSLQLTAPVAGQLGATTVPPSGLSVPFSTWD
QDHDLRRDKNCAKSLSGGWWFGTCSHSNLNGQYFRSIPQQRQKLKKGIFW
KTWRGRYYPLQATTMLIQPMAAEAAS
DKK1
(SEQ ID NO: 2)
VSATLNSVLNSNAIKNLPPPLGGAAGHPGSAVSAAPGILYPGGNKYQTID
NYQPYPCAEDEECGTDEYCASPTRGGDAGVQICLACRKRRKRCMRHAMCC
PGNYCKNGICVSSDQNHFRGEIEETITESFGNDHSTLDGYSRRTTLSSKM
YHTKGQEGSVCLRSSDCASGLCCARHFWSKICKPVLKEGQVCTKHRRKGS
HGLEIFQRCYCGEGLSCRIQKDHHQASNSSRLHTCQRH
EPHA2
(SEQ ID NO: 3)
KEVVLLDFAAAGGELGWLTHPYGKGWDLMQNIMNDMPIYMYSVCNVMSGD
QDNWLRTNWVYRGEAERIFIELKETVRDCNSFPGGASSCKETFNLYYAES
DLDYGTNFQKRLFTKIDTIAPDEITVSSDFEARHVKLNVEERSVGPLTRK
GEYLAFQDIGACVALLSVRVYYKKCPELLQGLAHFPETIAGSDAPSLATV
AGTCVDHAVVPPGGEEPRMHCAVDGEWLVPIGQCLCQAGYEKVEDACQAC
SPGFFKFEASESPCLECPEHTLPSPEGATSCECEEGFFRAPQDPASMPCT
RPPSAPHYLTAVGMGAKVELRWTPPQDSGGREDIVYSVTCEQCWPESGEC
GPCEASVRYSEPPHGLTRTSVTVSDLEPHMNYTFTVEARNGVSGLVTSRS
FRTASVSINQTEPPKVRLEGRSTTSLSVSWSIPPPQQSRVWKYEVTYRKK
GDSNSYNVRRTEGFSVTLDDLAPDTTYLVQVQALTQEGQGAGSKVHEFQT
LSPEGSGNL
LAMC2
(SEQ ID NO: 4)
TSRREVCDCNGKSRQCIFDRELHRQTGNGFRCLNCNDNTDGIHCEKCKNG
FYRHRERDRCLPCNCNSKGSLSARCDNSGRCSCKPGVTGARCDRCLPGFH
MLTDAGCTQDQRLLDSKCDCDPAGIAGPCDAGRCVCKPAVTGERCDRCRS
GYYNLDGGNPEGCTQCFCYGHSASCRSSAEYSVHKITSTFHQDVDGWKAV
QRNGSPAKLQWSQRHQDVFSSAQRLDPVYFVAPAKELGNQQVSYGQSLSE
DYRVDRGGRHPSAHDVILEGAGLRITAPLMPLGKTLPCGLTKTYTFRLNE
HPSNNWSPQLSYFEYRRLLRNLTALRIRATYGEYSTGYIDNVTLISARPV
SGAPAPWVEQCICPVGYKGQFCQDCASGYKRDSARLGPFGTCIPCNCQGG
GACDPDTGDCYSGDENPDIECADCPIGFYNDPHDPRSCKPCPCHNGFSCS
VMPETEEVVCNNCPPGVTGARCELCADGYFGDPFGEHGPVRPCQPCQCNN
NVDPSASGNCDRLTGRCLKCIHNTAGIYCDQCKAGYFGDPLAPNPADKCR
ACNCNPMGSEPVGCRSDGTCVCKPGFGGPNCEHGAFSCPACYNQVKIQMD
QFMQQLQRMEALISKAQGGDGVVPDTELEGRMQQAEQALQDILRDAQISE
GASRSLGLQLAKVRSQENSYQSRLDDLKMTVERVRALGSQYQNRVRDTHR
LITQMQLSLAESEASLGNTNIPASDHYVGPNGFKSLAQEATRLAESHVES
ASNMEQLTRETEDYSKQALSLVRKALHEGVGSGSGSPDGAVVQGLVEKLE
KTKSLAQQLTREATQAEIEADRSYQHSLRLLDSVSRLQGVSDQSFQVEEA
KRIKQKADSLSSLVTRHMDEFKRTQKNLGNWKEEAQQLLQNGKSGREKSD
QLLSRANLAKSRAQEALSMGNATFYEVESILKNLREFDLQVDNRKAEAEE
AMKRLSYISQKVSDASDKTQQAERALGSAAADAQRAKNGAGEALEISSEI
EQEIGSLNLEANVTADGALAMEKGLASLKSEMREVEGELERKELEFDTNM
DAVQMVITEAQKVDTRAKNAGVTIQDTLNTLDGLLHLMGM
SPON2
(SEQ ID NO: 5)
QPLGGESICSARAPAKYSITFTGKWSQTAFPKQYPLFRPPAQWSSLLGAA
HSSDYSMWRKNQYVSNGLRDFAERGEAWALMKEIEAAGEALQSVHEVFSA
PAVPSGTGQTSAELEVQRRHSLVSFVVRIVPSPDWFVGVDSLDLCDGDRW
REQAALDLYPYDAGTDSGFTFSSPNFATIPQDTVTEITSSSPSHPANSFY
YPRLKALPPIARVTLLRLRQSPRAFIPPAPVLPSRDNEIVDSASVPETPL
DCEVSLWSSWGLCGGHCGRLGTKSRTRYVRVQPANNGSPCPELEEEAECV
PDNCV
SSR2
(SEQ ID NO: 6)
EEGARLLASKSLLNRYAVEGRDLTLQYNIYNVGSSAALDVELSDDSFPPE
DFGIVSGMLNVKWDRIAPASNVSHTVVLRPLKAGYFNFTSATITYLAQED
GPVVIGSTSAPGQGGILAQREFDRRFSPH
GAL1
(SEQ ID NO: 7)
LRVRGEVAPDAKSFVLNLGKDSNNLCLHFNPRFNAHGDANTIVCNSKDGG
AWGTEQREAVFPFQPGSVAEVCITFDQANLTVKLPDGYEFKFPNRLNLEA
INYMAADGDFKIKCVAFD
GFRA1
(SEQ ID NO: 8)
DRLDCVKASDQCLKEQSCSTKYRTLRQCVAGKETNFSLASGLEAKDECRS
AMEALKQKSLYNCRCKRGMKKEKNCLRIYWSMYQSLQGNDLLEDSPYEPV
NSRLSDIFRVVPFISDVFQQVEHIPKGNNCLDAAKACNLDDICKKYRSAY
ITPCTTSVSNDVCNRRKCHKALRQFFDKVPAKHSYGMLFCSCRDIACTER
RRQTIVPVCSYEEREKPNCLNLQDSCKTNYICRSRLADFFTNCQPESRSV
SSCLKENYADCLLAYSGLIGTVMTPNYIDSSSLSVAPWCDCSNSGNDLEE
CLKFLNFFKDNTCLKNAIQAFGNGSDVTVWQPAFPVQTTTATTTTALRVK
NKPLGPAGSENEIPTHVLPPCANLQAQKLKSNVSGNTHLCISNGNYEKEG
LGASSHITTKSMAAPPSCGLSPLLVLVVTALSTLLSLTETS
LRRC15
(SEQ ID NO: 9)
YHGCPSECTCSRASQVECTGARIVAVPTPLPWNAMSLQILNTHITELNES
PFLNISALIALRIEKNELSRITPGAFRNLGSLRYLSLANNKLQVLPIGLF
QGLDSLESLLLSSNQLLQIQPAHFSQCSNLKELQLHGNHLEYIPDGAFDH
LVGLTKLNLGKNSLTHISPRVFQHLGNLQVLRLYENRLTDIPMGTFDGLV
NLQELALQQNQIGLLSPGLFHNNHINLQRLYLSNNHISQLPPSVFMQLPQ
LNRLTLFGNSLKELSPGIFGPMPNLRELWLYDNHISSLPDNVFSNLRQLQ
VLILSRNQISFISPGAFNGLTELRELSLHTNALQDLDGNVFRMLANLQNI
SLQNNRLRQLPGNIFANVNGLMAIQLQNNQLENLPLGIFDHLGKLCELRL
YDNPWRCDSDILPLRNWLLLNQPRLGTDTVPVCFSPANVRGQSLIIINVN
VAVPSVHVPEVPSYPETPWYPDTPSYPDTTSVSSTTELTSPVEDYTDLTT
IQVTDDRSVWGMTQAQSG
GRN
(SEQ ID NO: 10)
TRCPDGQFCPVACCLDPGGASYSCCRPLLDKWPTTLSRHLGGPCQVDAHC
SAGHSCIFTVSGTSSCCPFPEAVACGDGHHCCPRGFHCSADGRSCFQRSG
NNSVGAIQCPDSQFECPDFSTCCVMVDGSWGCCPMPQASCCEDRVHCCPH
GAFCDLVHTRCITPTGTHPLAKKLPAQRTNRAVALSSSVMCPDARSRCPD
GSTCCELPSGKYGCCPMPNATCCSDHLHCCPQDTVCDLIQSKCLSKENAT
TDLLTKLPAHTVGDVKCDMEVSCPDGYTCCRLQSGAWGCCPFTQAVCCED
HIHCCPAGFTCDTQKGTCEQGPHQVPWMEKAPAHLSLPDPQALKRDVPCD
NVSSCPSSDTCCQLTSGEWGCCPIPEAVCCSDHQHCCPQGYTCVAEGQCQ
RGSEIVAGLEKMPARRASLSHPRDIGCDQHTSCPVGQTCCPSLGGSWACC
QLPHAVCCEDRQHCCPAGYTCNVKARSCEKEVVSAQPATFLARSPHVGVK
DVECGEGHFCHDNQTCCRDNRQGWACCPYRQGVCCADRRHCCPAGFRCAA
RGTKCLRREAPRWDAPLRDPALRQLL
MUC1
(SEQ ID NO: 11)
APKPATVVTGSGHASSTPGGEKETSATQRSSVPSSTEKNAFNSSLEDPST
DYYQELQRDISEMFLQIYKQGGFLGLSNIKFRPGSVVVQLTLAFREGTIN
VHDVETQFNQYKTEAASRYNLTISDVSVSDVPFPFSAQSGAGVPG
CD147
(SEQ ID NO: 12)
AAGTVFTTVEDLGSKILLTCSLNDSATEVTGHRWLKGGVVLKEDALPGQK
TEFKVDSDDQWGEYSCVFLPEPMGTANIQLHGPPRVKAVKSSEHINEGET
AMLVCKSESVPPVTDWAWYKITDSEDKALMNGSESRFFVSS
CD320
(SEQ ID NO: 13)
AGPSSGSCPPTKFQCRTSGLCVPLTWRCDRDLDCSDGSDEEECRIEPCTQ
KGQCPPPPGLPCPCTGVSDCSGGTDKKLRNCSRLACLAGELRCTLSDDCI
PLTWRCDGHPDCPDSSDELGCGTNEILPEGDATTMGPPVTLESVTSLRNA
TTMGPPVTLESVPSVGNATSSSAGDQSGSPTAYG
CDH3
(SEQ ID NO: 14)
EPCRAVFREAEVTLEAGGAEQEPGQALGKVFMGCPGQEPALFSTDNDDFT
VRNGETVQERRSLKERNPLKIFPSKRILRRHKRDWVVAPISVPENGKGPF
PQRLNQLKSNKDRDTKIFYSITGPGADSPPEGVFAVEKETGWLLLNKPLD
REEIAKYELFGHAVSENGASVEDPMNISIIVTDQNDHKPKFTQDTFRGSV
LEGVLPGTSVMQMTATDEDDAIYTYNGVVAYSIHSQEPKDPHDLMFTIHR
STGTISVISSGLDREKVPEYTLTIQATDMDGDGSTTTAVAVVEILDANDN
APMFDPQKYEAHVPENAVGHEVQRLTVTDLDAPNSPAWRATYLIMGGDDG
DHFTITTHPESNQGILTTRKGLDFEAKNQHTLYVEVTNEAPFVLKLPTST
ATIVVHVEDVNEAPVFVPPSKVVEVQEGIPTGEPVCVYTAEDPDKENQKI
SYRILRDPAGWLAMDPDSGQVTAVGTLDREDEQFVRNNIYEVMVLAMDNG
SPPTTGTGTLLLTLIDVNDHGPVPEPRQITICNQSPVRQVLNITDKDLSP
HTSPFQAQLTDDSDIYWTAEVNEEGDTVVLSLKKFLKQDTYDVHLSLSD
HGNKEQLTVIRATVCDCHGHVETCPGPWKGG
HER2
(SEQ ID NO: 15)
TQVCTGTDMKLRLPASPETHLDMLRHLYQGCQVVQGNLELTYLPTNASLS
FLQDIQEVQGYVLIAHNQVRQVPLQRLRIVRGTQLFEDNYALAVLDNGDP
LNNTTPVTGASPGGLRELQLRSLTEILKGGVLIQRNPQLCYQDTILWKDI
FHKNNQLALTLIDTNRSRACHPCSPMCKGSRCWGESSEDCQSLTRTVCAG
GCARCKGPLPTDCCHEQCAAGCTGPKHSDCLACLHFNHSGICELHCPALV
TYNTDTFESMPNPEGRYTFGASCVTACPYNYLSTDVGSCTLVCPLHNQEV
TAEDGTQRCEKCSKPCARVCYGLGMEHLREVRAVTSANIQEFAGCKKIFG
SLAFLPESFDGDPASNTAPLQPEQLQVFETLEEITGYLYISAWPDSLPDL
SVFQNLQVIRGRILHNGAYSLTLQGLGISWLGLRSLRELGSGLALIHHNT
HLCFVHTVPWDQLFRNPHQALLHTANRPEDECVGEGLACHQLCARGHCWG
PGPTQCVNCSQFLRGQECVEECRVLQGLPREYVNARHCLPCHPECQPQNG
SVTCFGPEADQCVACAHYKDPPFCVARCPSGVKPDLSYMPIWKFPDEEGA
CQPCPINCTHSCVDLDDKGCPAEQRASPLT
IGFBP2
(SEQ ID NO: 6)
EVLFRCPPCTPERLAACGPPPVAPPAAVAAVAGGARMPCAELVREPGCGC
CSVCARLEGEACGVYTPRCGQGLRCYPHPGSELPLQALVMGEGTCEKRRD
AEYGASPEQVADNGDDHSEGGLVENHVDSTMNMLGGGGSAGRKPLKSGMK
ELAVFREKVTEQHRQMGKGGKHHLGLEEPKKLRPPPARTPCQQELDQVLE
RISTMRLPDERGPLEHLYSLHIPNCDKHGLYNLKQCKMSLNGQRGECWCV
NPNTGKLIQGAPTIRGDPECHLFYNEQQEARGVHTQRMQ
LRP10
(SEQ ID NO: 17)
HPDRIIFPNHACEDPPAVLLEVQGTLQRPLVRDSRTSPANCTWLILGSKE
QTVTIRFQKLHLACGSERLTLRSPLQPLISLCEAPPSPLQLPGGNVTITY
SYAGARAPMGQGFLLSYSQDWLMCLQEEFQCLNHRCVSAVQRCDGVDACG
DGSDEAGCSSDPFPGLTPRPVPSLPCNVTLEDFYGVFSSPGYTHLASVSH
PQSCHWLLDPHDGRRLAVRFTALDLGFGDAVHVYDGPGPPESSRLLRSLT
HFSNGKAVTVETLSGQAVVSYHTVAWSNGRGFNATYHVRGYCLPWDRPCG
LGSGLGAGEGLGERCYSEAQRCDGSWDCADGTDEEDCPGCPPGHFPCGAA
GTSGATACYLPADRCNYQTFCADGADERRCRHCQPGNFRCRDEKCVYETW
VCDGQPDCADGSDEWDCSYVLPRK
SPINT2
(SEQ ID NO: 18)
ADRERSIHDFCLVSKVVGRCRASMPRWWYNVTDGSCQLFVYGGCDGNSNN
YLTKEECLKKCATVTENATGDLATSRNAADSSVPSAPRRQDSEDHSSDMF
NYEEYCTANAVTGPCRASFPRWYFDVERNSCNNFIYGGCRGNKNSYRSEE
ACMLRCFRQQENPPLPLGSKV
SUSD2
(SEQ ID NO: 19)
QESCSMRCGALDGPCSCHPTCSGLGTCCLDFRDFCLEILPYSGSMMGGKD
FVVRHFKMSSPTDASVICRFKDSIQTLGHVDSSGQVHCVSPLLYESGRIP
FTVSLDNGHSFPRAGTWLAVHPNKVSMMEKSELVNETRWQYYGTANTSGN
LSLTWHVKSLPTQTITIELWGYEETGMPYSQEWTAKWSYLYPLATHIPNS
GSFTFTPKPAPPSYQRWRVGALRIIDSKNYAGQKDVQALWTNDHALAWHL
SDDFREDPVAWARTQCQAWEELEDQLPNFLEELPDCPCTLTQARADSGRF
FTDYGCDMEQGSVCTYHPGAVHCVRSVQASLRYGSGQQCCYTADGTQLLT
ADSSGGSTPDRGHDWGAPPFRTPPRVPSMSHWLYDVLSFYYCCLWAPDCP
RYMQRRPSNDCRNYRPPRLASAFGDPHFVTFDGTNFTFNGRGEYVLLEAA
LTDLRVQARAQPGTMSNGTETRGTGLTAVAVQEGNSDVVEVRLANRTGGL
EVLLNQEVLSFTEQSWMDLKGMFLSVAAGDRVSIMLASGAGLEVSVQGPF
LSVSVLLPEKFLTHTHGLLGTLNNDPTDDFTLHSGRVLPPGTSPQELFLF
GANWTVHNASSLLTYDSWFLVHNFLYQPKHDPTFEPLFPSETTLNPSLAQ
EAAKLCGDDHFCNFDVAATGSLSTGTATRVAHQLHQRRMQSLQPVVSCGW
LAPPPNGQKEGNRYLAGSTIYFHCDNGYSLAGAETSTCQADGTWSSPTPK
CQPGRSYA
CST2
(SEQ ID NO: 20)
WSPQEEDRIIEGGIYDADLNDERVQRALHFVISEYNKATEDEYYRRLLRV
LRAREQIVGGVNYFFDIEVGRTICTKSQPNLDTCAFHEQPELQKKQLCSF
QIYEVPWEDRMSLVNSRCQEA

In a further embodiment, the antibody detection marker is a protein, such as those disclosed above, that is in its native form. As disclosed in the accompanying examples, the inventors utilized a eukaryotic expression system to generate conformation-carrying tumor antigens that are properly folded and contain non-continuous epitopes for use in the detection of autoantibodies. The protein may be used in any suitable format; in one non-limiting embodiment, the protein may be an Fc fusion protein.

In all of the above embodiments, the antibody detection reagents can be labeled with a detectable label. In one embodiment, the detectable labels for reagents to detect autoantibodies against one protein are distinguishable from the detectable labels to detect autoantibodies against the other protein. Methods for detecting the label include, but are not limited to spectroscopic, photochemical, biochemical, immunochemical, physical or chemical techniques. Any suitable detectable label can be used.

The compositions can be stored frozen, in lyophilized form, or as a solution. In one embodiment, the compositions can be placed on a solid support, such as in a microarray or microplate format; this embodiment facilitates use of the compositions in various detection assays. For example, anti-IgG can be used to precoat the wells of a microwell plate and the antibody detection reagents (such as the proteins discussed herein) can be added to the precoated wells.

In a second aspect, the present invention provides methods for detecting breast cancer or breast cancer recurrence, comprising contacting a bodily fluid sample from a subject at risk of having breast cancer or breast cancer recurrence with one or more reagents for detecting autoantibodies against one or more of human ANGTPL4, DKK1, EPHA2, LAMC2, SPON2, SSR2, GAL1, GFRA1, LRRC15, CD147, CD320, CDH3, LRP10, SPINT2, SUSD2, and CST2, wherein the presence of autoantibodies against the one or more proteins correlates with a likelihood of the subject having breast cancer or breast cancer recurrence.

In one embodiment, the composition includes reagents for detecting human autoantibodies against at least two proteins selected from the group consisting of human ANGPTL4, DKK1, EPHA2, GAL1, LAMC2, SPON2, CST2, SPINT2 and SSR2.

As will be understood by those of skill in the art, the methods may include the use of additional antibody detection markers and controls as is appropriate for an intended use of the composition. In one non-limiting embodiment, the compositions may further comprise reagents for detecting antibodies against one or both of mucin-1(MUC1), HER-2 (41), IGFBP2, and GRANULIN.

In another embodiment of the methods of the invention, the compositions comprise or consist of reagents for detecting human autoantibodies against one of the following marker sets:

### ANGPTL4, DKK1, GAL1, GFRA1, GRANULIN, LRRC15, and MUC1;

### ANGPTL4, DKK1, GAL1, GRANULIN, LRRC15, and MUC1;

### ANGPTL4, DKK1, GAL1, and LRRC15;

### ANGPTL4, DKK1, GAL1, GFRA1, and LRRC15;

### DKK1, GAL1, GFRA1, GRANULIN, LRRC1, and 5 MUC1;

### ANGPTL4, DKK1, GAL1, GFRA1, GRANULIN, and LRRC15;

### DKK1, GAL1, GRANULIN, LRRC15, and MUC1;

### DKK1, GAL1, GFRA1, GRANULIN, and LRRC15;

### DKK1, GAL1, GFRA1, LRRC15, and MUC1;

### ANGPTL4, DKK1, GAL1, GRANULIN, and LRRC15;

### DKK1, GAL1, GFRA1, and LRRC15;

### DKK1, GAL1, GRANULIN, and LRRC15;

### ANGPTL4, DKK1, GAL1, LRRC15, and MUC1;

### DKK1, GAL1, and LRRC15;

### ANGPTL4, GAL1, LRRC15, and MUC1;

### GAL1, GFRA1, LRRC15, and MUC1;

### GAL1, GFRA1, and LRRC15;

### ANGPTL4, GAL1, and LRRC15;

### DKK1, GAL1, LRRC15, and MUC1;

### ANGPTL4, GAL1, GFRA1, and LRRC15;

### GAL1, LRRC15, and MUC1;

### ANGPTL4, GAL1, GFRA1, LRRC15, and MUC1;

### ANGPTL4, GAL1, and GFRA1;

### DKK1, GAL1, and GFRA1; and

### GAL1, GFRA1, and MUC1.

In another embodiment, the compositions comprise or consist of reagents for detecting human autoantibodies against ANGPTL4, DKK1, GAL1, MUC1, GFRA1, GRN and LRRC15.

The antibody detection markers may be any suitable reagents that can be used to detect antibodies against the recited proteins, including but not limited to the recited protein, a secreted version of the protein (such as a native secreted form of the protein), or an extracellular domain of the protein. Secreted proteins are more easily delivered from tumor cells to lymph nodes, where interactions of immune cells take place resulting in abundant high-affinity antibodies. Membrane surface proteins are commonly released in a soluble form from tumor cells through metalloproteinase-dependent cleavage. The shed proteins are more easily transferred to the lymph nodes than intracellular protein. Thus, in one embodiment the antibody detection marker is a secreted or membrane portion of the recited protein. Exemplary amino acid sequences of the secreted or membrane portion of the recited proteins are as disclosed herein.

In another embodiment, the antibody detection marker comprises or consists of a composition of the invention.

The contacting can be carried out under any suitable conditions for promoting binding between the autoantibodies in the bodily fluid sample and the reagent to forma binding complex that can be detected. Appropriate such conditions can be determined by those of skill in the art based on the intended assay, in light of the teachings herein. Similarly, any suitable additional steps can be used in the methods, such as one or more wash or other steps to remove unbound reagents.

Any suitable detection technique can be used, including but not limited to enzyme linked immunosorbent assays (ELISA), bead based assay platforms such as the Luminex systems, 2-D array based assay platforms such as SearchLight®, and the Inanovate® ‘Longitudinal Assay Screening’ platform which may be capable of quantitating all the listed breast cancer biomarker from patient samples at their clinically relevant concentrations in a single test and dilution. In one embodiment, the compositions can be placed on a solid support, such as in a microarray, glass slide, membrane, microplate format or beads. The embodiment facilitates use of the compositions. Exemplary such assays are provided in the examples.

Similarly, any suitable bodily fluid can be used, including but not limited to a serum sample, plasma sample or blood sample from the subject. The subject may be any subject at risk of breast cancer, such as a human subject.

In a further embodiment, method identifies the subject as likely to have breast cancer, and wherein the method further comprises treating the subject with an amount of a therapeutic sufficient to treat the breast cancer.

In one non-limiting embodiment of any of the above embodiments, ANGPTL4, DKK1, GAL1, and MUC1 autoantibody response are correlated with BCa; and autoantibody responses against GFRA1, GRN and LRRC15 are inversely correlated with BCa.

In one specific embodiment, the reagents include ANGPTL4, DKK1, GAL1, MUC1, GFRA1, GRN and LRRC15; where, autoantibody responses against GFRA1, GRN and LRRC15 are inversely correlated with BCa. As detailed in the examples, when the autoantibody responses against the 7 antigens were added to the base model, including age, body mass index (BMI), race and current smoking status, the assay had the following diagnostic capabilities: c-stat (95% CI), 0.82 (0.78 to 0.86); sensitivity, 73%; specificity, 76%; and PLR (95% CI), 3.04 (2.34 to 3.94). The model was calibrated across risk deciles (Hosmer-Lemeshow, p=0.13) and performed well in specific subtypes of BCa including estrogen receptor positive, HER-2 positive, invasive, in situ and tumor sizes >1 cm. Diagnostic capabilities of other exemplary marker sets are provided in Table 5.

In a third aspect, the invention provides methods for treating a subject with breast cancer, comprising:

(a) testing a bodily fluid sample from a subject at risk of breast cancer, and identifying candidate subjects that:
(i) have autoantibodies against at least one of ANGPTL4, DKK1, GAL1, MUC1, GFRA1, GRN and LRRC15; and/or

(b) do not have autoantibodies against GFRA1, GRN and/or LRRC15; and

(b) treating the candidate subjects with an amount of a therapeutic sufficient to treat the breast cancer.

### Example 1

Breast cancer (BCa) patients elicit an autoantibody response against cancer proteins, which reflects and amplifies the cellular changes associated with tumorigenesis. Detection of autoantibodies in plasma may provide a minimally invasive mechanism for early detection of BCa. To identify cancer proteins that elicit a humoral response, we generated a cDNA library enriched for BCa genes that encode membrane and secreted proteins, which are more likely to induce an antibody response compared to intracellular proteins. To generate conformation-carrying antigens that are efficiently recognized by patients' antibodies, a eukaryotic expression strategy was established. Plasma from 200 BCa patients and 200 age-matched healthy controls were measured for autoantibody activity against 20 different antigens designed to have conformational epitopes using ELISA. A conditional logistic regression model was used to select a combination of autoantibody responses against the 20 different antigens to classify BCa patients from healthy controls. The best combination included ANGPTL4, DKK1, GAL1, MUC1, GFRA1, GRN and LRRC15; however, autoantibody responses against GFRA1, GRN and LRRC15 were inversely correlated with BCa. When the autoantibody responses against the 7 antigens were added to the base model, including age, BMI, race and current smoking status, the assay had the following diagnostic capabilities: c-stat (95% CI), 0.82 (0.78 to 0.86); sensitivity, 73%; specificity, 76%; and PLR (95% CI), 3.04 (2.34 to 3.94). The model was calibrated across risk deciles (Hosmer-Lemeshow, p=0.13) and performed well in specific subtypes of BCa including estrogen receptor positive, HER-2 positive, invasive, in situ and tumor sizes >1 cm.

### INTRODUCTION

For patients with breast cancer (BCa), early and personalized diagnosis is crucial for optimizing treatments leading to long-term survival. Although mammography is the most widely used method to detect BCa, approximately 20% of screening mammograms result in a false negative diagnosis largely due to high breast density (1). Additionally, 1 in 10 women who get a mammogram will need additional imaging (2). Yet, the overwhelming majority of these women will not have BCa, as only 2 to 4 of every 1,000 screening mammograms leads to a cancer diagnosis (3). Therefore, there is an urgent clinical need to develop a novel, minimally invasive diagnostic strategy for the early diagnosis of BCa.

At present, there is no established tumor marker that is secreted into the peripheral circulation that can be measured by a blood test for the diagnosis of BCa. Currently, tumor markers that are accepted in clinical practice are tissue-based prognostic markers, such as the estrogen receptor (ER), HER-2 amplification, 21-gene Oncotype DX and 70-gene MammaPrint (6-12). All require an invasive biopsy or surgical procedure to acquire tumor tissue for assessment, bearing a heavy burden on patients. Serum tumor markers are valuable tools that allow minimally invasive procedures for sampling to promote the early diagnosis of cancer as well as following the prognosis after treatment (4, 5). However, tumor markers produced by tumor cells usually have relatively low concentrations in the peripheral circulation, especially in early stage disease.

Here we report the use of a molecular approach to identify tumor antigen candidates that elicit an antibody response in BCa patients. Previously, we generated a BCa cDNA library from membrane-associated polyribosomal (MAP) RNA, which encodes secreted and membrane proteins, and subtracted the library with RNA from normal tissues (29). Secreted proteins are more easily delivered from tumor cells to lymph nodes, where interactions of immune cells take place resulting in abundant high-affinity antibodies. Membrane surface proteins are commonly released in a soluble form from tumor cells through metalloproteinase-dependent cleavage. The shed proteins are more easily transferred to the lymph nodes than intracellular proteins (30, 31). Consequently, the obtained subtracted library, referred to as the membrane-associated polyribosomal cDNA library (MAPcL), is enriched with clones encoding membrane and secreted TAA that are highly abundant in BCa and should preferentially induce an antibody response in patients (29). In addition, we have established a method for producing recombinant antigens as Fc fusion proteins designed to have native conformations, which is essential for the expression of membrane and secreted proteins that may induce an antibody response in patients.

We have developed a conformation-carrying antigen ELISA-based strategy to discriminate between BCa and healthy patients by the detection of autoantibodies against a panel of TAAs. Twenty antigens were selected from the most abundant genes represented in the MAPcL, and Fc fusion proteins were generated. Blood was collected from 200 newly diagnosed BCa patients and 200 healthy women as age-matched controls. The 400 plasma samples were screened for the presence of autoantibodies against the 20 different MAPcL-derived antigens using ELISA. A combination of seven antigens with patient demographics yielded the best positive likelihood ratio to discriminate between healthy and BCa patients.

### Materials and Methods

### Plasmid Construction

For production of MAPcL-rabbit Fc-tagged antigens, two constructs, pSecTag2 (Invitrogen, Carlsbad, Calif.) and pFUSE-rIgG-Fc1 (InvivoGen, San Diego, Calif.), were both utilized to generate the 20 MAPcL-rFc expression constructs because of restriction site availability for cloning. pSecTag2 was modified by amplifying the Fc portion of rabbit IgG using primers 5′-CCGGATATCAGCAAGCCCACGTGCCCACC-3′ (SEQ ID NO: 21) and 5′-AAGGAAAAAAGCGGCCGCTC-ATTTACCCGGAGAGCGGGAG-3′ (SEQ ID NO: 22) (Integrated DNA Technologies, Coralville, Iowa) using pFUSE-rIgG-Fc1 as a template. The rFc PCR product was digested with EcoRV and NotI and inserted into pSecTag2, referred to as pSecTag2-rFc, which contains an IgK signal sequence for secretion. The pFUSE-rIgG-Fc1 contains an IL2 signal sequence. To keep the signal sequence consistent between the two plasmids, the IgK leader sequence was amplified via PCR using pSecTag2 as a template. The IL2 leader sequence was then replaced with the IgK signal sequence, creating pFUSE-IgK-rFc.

The accession numbers of the 20 MAPcL genes used as templates for cloning and predicted signal sequences are indicated in Table 1. The signal sequences of each encoded protein were determined using SignalP (32, 33). If a protein contained a transmembrane domain, only the encoded extracellular portion was included. The transmembrane domains were predicted using the TMHMM database (34). The amino acid numbers encoded by the cloned fragment are shown in Table 1. ANGPTL4, CDH3, DKK1, SPON2, SSR2, CST2, GFRA1 and GAL1 were custom cloned into pSecTag2-rFc using the SfiI and KpnI restriction sites (Genscript, Piscataway, N.J.). EPHA2, IGFBP2 and LAMC2 were custom cloned into pSecTag2-rFc using the KpnI and BamHI restriction sites. GRN, MUC1 and LRRC15 were custom cloned into pSecTag2-rFc using the SfiI and BamHI restriction sites. HER-2, LRP10, SPINT2 and SUSD2 were cloned into pFUSE-IgK-rFc using the SfiI and XhoI restriction sites. CD147 was cloned into pFUSE-IgK-rFc using the BamHI and SacII restriction sites. CD320 was cloned into pFUSE-IgK-rFc using the EcoRI and XhoI restriction sites.

TABLE 1
MAPcL Candidates for Generation of rFc Fusion Proteins

| | | Signal Sequence* | Encoded Amino |
|---|---|---|---|
| Gene from MAPcL | Accession # | Amino Acids | Acid Fragment† |
| ANGPTL4 (angiopoietin-like 4) | NM_139314 | 1-30 | 31-406 |
| CD147 | NM_198589 | 1-21 | 22-162 |
| CD320 | NM_016579 | 1-46 | 47-230 |
| CDH3 (cadherin 3) | NM_001793 | 1-24 | 25-654 |
| CST2 (cystatin SA) | NM_001322 | 1-20 | 21-141 |
| DKK1 (dickkopf WNT signaling | NM_12242 | 1-28 | 29-266 |
| pathway inhibitor 1) | | | |
| EPHA2 (EPH receptor A2) | NM_004431 | 1-26 | 27-535 |
| GAL1 (lectin, galactoside-binding, | NM_002305 | 1-17 | 18-135 |
| soluble, 1) | | | |
| GFRA1 (GPI-linked anchor protein) | AF038421 | 1-24 | 25-465 |
| GRN (granulin) | NM_002087 | 1-17 | 18-593 |
| HER-2 | NM_004448 | 1-22 | 23-652 |
| IGFBP2 (insulin-like growth factor | NM_000597 | 1-39 | 40-328 |
| binding protein 2) | | | |
| LAMC2 (laminin, gamma 2) | NM_005562 | 1-21 | 22-1111 |
| LRP10 (low density lipoprotein receptor- | NM_014045 | 1-16 | 17-440 |
| related protein 10) | | | |
| LRRC15 (leucine rich repeat containing | NM_001135057 | 1-27 | 28-544 |
| 15) | | | |
| MUC1 (mucin 1) | NM_002456 | 1-22 | 23-167 |
| SPINT2 (serine peptidase inhibitor, | NM_021102 | 1-27 | 28-198 |
| Kunitz type, 2) | | | |
| SPON2 (spondin 2) | NM_012445 | 1-26 | 27-331 |
| SSR2 (signal sequence receptor, beta | NM_003145 | 1-17 | 18-146 |
| (translocon-associated protein beta)) | | | |
| SUSD2 (sushi domain containing 2) | NM_019601 | 1-27 | 28-785 |

*The signal sequences of each encoded protein were determined using SignalP (32, 33) and were not included in the expression constructs.
†The amino acid numbers indicate the encoded portion of the proteins cloned between the Ig□ signal sequence and the Fc portion of rabbit IgG to generate the secreted MAPcL-rFc fusion proteins.

For production of His-tagged HER-2, HER-2 was amplified via PCR using primers 5′-CCCAAGCTTGCAGCACCCAAGTGTGCACCGGCAC-3′ (SEQ ID NO: 23) and 5′-GTGCTCGAGTCACGTC-AGAGGGCTGGCTCTCTGCTCG-3′(SEQ ID NO: 24). The product was digested with HindIII and XhoI and cloned directionally into the pET-28a expression vector.

### Cell Culture

293T and SKBR3 cell lines were cultured in DMEM with 10% FBS. Cultures were maintained at 37° C. with 5% CO₂ in a humidified incubator. All cell lines were authenticated and tested negatively for mycoplasma.

### Protein Production

The MAPcL-rFc fusion proteins were produced in 293T cells. Briefly, 293T cells were transfected using Effectene (Qiagen, Valencia, Calif.) according to manufacturer's specifications. During transfection, the cells were cultured in DMEM with 2% FBS. Supernatants containing the secreted fusion proteins were harvested, centrifuged to clear cell debris and supplemented with 0.1% sodium azide. His-HER-2 was produced in E. coli BL21 (Invitrogen, Carlsbad, Calif.) and purified using IMAC affinity chromatography.

### Sandwich ELISA

Microtiter plates (Nalge Nunc, Rochester, N.Y.) were coated overnight with 2 μg/ml goat anti-rabbit Fc (Jackson Immunoresearch, West Grove, Pa.) diluted with phosphate buffered saline. The supernatants containing the rFc fusion proteins were diluted 1:3 serially in standard blocking buffer (0.5% bovine serum albumin and 0.1% sodium azide in phosphate buffered saline). Plates were washed once, and the serially diluted supernatants were transferred to the microtiter plates. Rabbit IgG of known concentration was diluted similarly and added to one row of the microtiter plate in order to quantify the amount of fusion protein present in the culture media. After incubating for two hours, plates were washed twice and 50 μl of HRP-conjugated goat anti-rabbit IgG (Jackson Immunoresearch, West Grove, Pa.) diluted 1:3000 in standard blocking buffer with 0.05% Tween 20 added. After a 2-hour incubation, plates were washed 4 times and developed with 100 μl/well of TMB substrate (Pierce, Rockford, Ill.). The development reaction was stopped after five minutes with 50 μl/well of 2N H₂SO₄, and the absorbance was measured at 450 nm to determine the concentration. The absorbance at 690 nm was subtracted to remove background signal.

### Antibody Recognition of Conformational Versus Denatured HER-2 Protein

For the conformational HER-2 assay, microtiter plates were coated with 2 μg/ml goat anti-rabbit Fc (Jackson Immunoresearch, West Grove, Pa.) in PBS overnight. HER-2-ECD-rFc was then added to each well, 100 μl/well. For denatured HER-2, microtiter plates were coated with 2 μg/ml His-HER-2-ECD in PBS overnight.

Three HER-2 antibodies were used in the assay: anti-HER-2 3F27 (US Biological, Swampscott, Mass.), anti-HER-2 3F32 (US Biological, Swampscott, Mass.) and Herceptin (Genentech, South San Francisco, Calif.). Each antibody was diluted to 1 μg/ml in standard blocking buffer with 0.05% Tween 20. The antibodies were then serially diluted. After washing once, 50 μl/well of the serially diluted antibodies was added to the plates and incubated for 2 hours at room temperature. The plates were washed three times, and species appropriate HRP-conjugated secondary antibodies were added at a 1:3000 dilution. Plates were washed four times and developed with 100 μl/well TMB substrate for five minutes. Development was stopped with 50 μl/well 2N H₂SO₄. Absorbance was measured at 450 nm, and the 690 nm absorbance was subtracted to account for background.

The same antibodies were used to stain HER-2 in SKBR3 BCa cells via flow cytometry. SKBR3 cells were detached from dish using Cell Dissociation Solution Non-enzymatic 1× (Sigma, St. Louis, Mo., catalog # C5914). 2×10⁵ cells were incubated with 0.5 μg/ml of each antibody for 1 hour at room temperature. The cells were then washed, and a 1:200 dilution of PE-conjugated antibody for the appropriate species was added. The cells were again washed, resuspended in FACS buffer (PBS with 5% bovine serum albumin and 0.1% sodium azide) and analyzed by flow cytometry.

### Competition of Herceptin Binding

Microtiter plates were coated with 4 μg/ml goat anti-rabbit Fc and incubated overnight. After one wash, 100 μl/well HER-2-ECD-rFc was added to each well and incubated overnight. HER-2-Fc and CD30-Fc chimeric proteins (R&D Systems, Minneapolis, Minn.) were serially diluted from a starting concentration of 10 ug/ml. Herceptin was added to a final concentration of 10 ng/ml in each of the serial chimeric protein dilutions. Plates were washed twice, and 50 μl/well of chimeric protein/Herceptin mixture was applied to the plate. Plates were then washed three times, and a 1:3000 dilution of HRP goat anti-human IgG was applied to each well, 50 μl/well. After four washes, 100 μl/well TMB substrate was added to each well. Development was stopped with 50 μl/well 2N H2SO4 after 5 minutes. Absorbance was measured at 450 nm with 690 nm absorbance subtracted.

### Patients

The inclusion criteria for cases were women over 30 years of age that were newly diagnosed with BCa (any type) at Sanford Health, Sioux Falls, S. Dak. Patients were asked to provide one extra 10 ml EDTA tube of blood prior to mastectomy, lumpectomy, radiation therapy, chemotherapy or other treatment. Case subjects were excluded only if they had a previous history of cancer of any kind. Healthy control subjects had a negative mammogram within six months before the blood draw. Healthy subjects were excluded if there was a history of previous cancer of any kind or a history of autoimmune disease. All patients provided written informed consent, and the Sanford Health IRB approved the study protocol. Blood samples from 200 BCa patients were collected from Oct. 8, 2009 to Apr. 17, 2012. In addition, 200 age-matched healthy control blood samples were collected from Oct. 16, 2009 to Jan. 19, 2011. See Table 2 for enrolled patients' characteristics.

TABLE 2
Patient Clinical and Pathological Characteristics

| | N = 200 | |
|---|---|---|
| Patients with Breast Cancer | | |
| Age: Mean (SD) | 58.9 | (11.4) |
| White Race: n (%) | 193 | (97%) |
| BMI [kg/m2]: Mean (SD) | 29.7 | (6.6) |
| Smoking Status: n (%) | | |
| Current | 22 | (11%) |
| Never | 120 | (60%) |
| Past | 58 | (29%) |
| Family History Yes: n (%) | 114 | (58%) |
| Tumor Type: n (%) | | |
| Invasive | 148 | (74%) |
| in situ | 52 | (26%) |
| Histology: n (%) | | |
| Ductal and Lobular | 3 | (2%) |
| Ductal | 173 | (87%) |
| Lobular | 21 | (11%) |
| Other | 2 | (1%) |
| ER Positive: n (%) | 171 | (86%) |
| PR Positive: n (%) | 147 | (74%) |
| HER-2 Amplification: n (%) | | |
| Negative | 156 | (78%) |
| Positive | 33 | (17%) |
| Unknown | 11 | (6%) |
| Triple Negative Yes: n (%) | 18 | (12%) |
| Tumor Max Dimension [cm]: n (%) | | |
| ≤1 | 66 | (36%) |
| >1 to ≤2 | 65 | (35%) |
| >2 | 53 | (29%) |
| Lymph Node Involvement: n (%) | 47 | (24%) |
| Age-Matched Controls | | |
| with Negative Mammogram | | |
| Age: Mean (SD) | 58.8 | (11.3) |
| White Race: n (%) | 192 | (97%) |
| BMI [kg/m2]: Mean (SD) | 27.1 | (5.5) |
| Smoking Status: n (%) | | |
| Current | 7 | (4%) |
| Never | 125 | (63%) |
| Past | 67 | (34%) |

### Serum Collection

Blood was collected in a 10 ml EDTA tube and centrifuged at 2000×g for 10 minutes. Plasma was removed from the tube, aliquoted and stored at −80 degrees Celsius until screening for the presence of autoantibodies.

### Conformation-Carrying Antigen ELISA

Microtiter plates (Nalge Nunc, Rochester, N.Y.) were coated overnight with 4 μg/ml goat anti-rabbit Fc (Jackson Immunoresearch, West Grove, Pa.) in phosphate buffered saline. Plates were washed once, and 100 μl/well of MAPcL-rFc fusion protein was added. Plates were incubated for 2 hours and washed twice. The plates were then coated with 50 μl/well of optimized blocking buffer (phosphate buffered saline with 0.5% bovine serum albumin, 0.2% dry milk, 0.1% polyvinylpyrrolidone, 20 mM L-Glutamine, 20 mM L-Arginine, 0.1% sodium azide, 10% goat serum, and 0.05% Tween 20). The plates were incubated for 1 hour at 37° C. and washed once. Serum samples diluted 1:100 in optimized blocking buffer were added and incubated for 2 hours at room temperature. Plates were then washed three times, and autoantibodies were detected using an HRP-conjugated goat anti-human IgG (Jackson Immunoresearch, West Grove, Pa.) diluted 1:3000 in standard blocking buffer with 0.05% Tween 20. Plates were incubated for 1 hour at room temperature, washed four times and developed with 100 μl/well of TMB substrate (Pierce, Rockford, Ill.) for 15 minutes. Development was stopped with 50 μl/well 2N H₂SO₄, and the absorbance was measured at 450 nm. The absorbance at 690 nm was subtracted to remove background signal. Each 96-well plate included 14 samples from BCa subjects and 14 samples from normal mammogram subjects. Each sample was tested in triplicate within the same plate. One row in each plate was subjected only to blocking buffer as a negative control for the ELISA.

### Statistical Methods

Controls were individually matched to 200 BCa patients 1:1 within a 3-year age window using a greedy caliper matching algorithm (35) while blinded to assay data. For each subject the antigen level was transformed by subtracting the mean of the blocking buffer from the mean of the triplicate measurements. If the difference was less than zero, it was set to zero, and the square root was taken to yield a more symmetrical distribution.

Differences in demographics and autoantibody responses between BCa patients and controls were tested using two-sample t-test and Chi-squared test for continuous and categorical data, respectively. The incremental improvement to the c-statistic (i.e. concordance index, area under the receiver operating characteristic (ROC) curve) was tested by adding the autoantibody response to each antigen to a logistic regression model that already included age, BMI, race, and current smoking status. The model calibration was tested using the Hosmer-Lemeshow goodness-of-fit measure, which constructs a Chi-squared statistic by comparing the predicted and observed number of cases by probability decile (36).

After assessing the individual antigens, a multivariable conditional logistic regression analysis with strata for age-matching was used to determine the subset of antigens that minimized Akaike's Information Criterion (37); all models were adjusted for BMI, race, and current smoking status. Exploratory subgroup analyses were performed to determine if the multivariable subset of antigens performed differently in a particular type of BCa. The multivariable model was tested in the following subgroups: invasive, in situ, ER positive, tumor maximum dimension >1 cm, lymph node involvement, and HER-2 positive. The critical level alpha was set to ≤0.05/20 antigens=0.0025 using the Bonferroni correction. SAS® (Cary, N.C.) version 9.3 software was used for all analyses.

### Results

Generation of Tumor-Associated Antigens Designed to have Native Conformations

To identify TAAs that elicit a humoral response in patients, candidate genes that encode membrane and secreted proteins were selected from the most abundant genes represented in the MAPcL. Because only 10% of epitopes on proteins are in a linear continuous sequence (24), we utilized a eukaryotic expression system to generate conformation-carrying tumor antigens that are properly folded and contain noncontinuous epitopes for use in the detection of autoantibodies. Sequences encoding the extracellular domains (ECD) or the secreted proteins without the signal sequence of the candidate MAPcL genes were cloned 5′ of the Fc region of rabbit IgG (rFc) into the pSecTag2-rFc vector or pFUSE-IgK-rFc, depending on restriction enzyme cloning sites. The IgK leader sequence contained in the vectors directs the fusion proteins to be secreted. The vectors encoding the fusion proteins were transiently transfected into 293T cells, and the corresponding fusion proteins were secreted into the media. Production of the secreted fusion proteins was confirmed using a sandwich ELISA, and the concentrations were determined by comparison to an established CD147-rFc standard (data not shown).

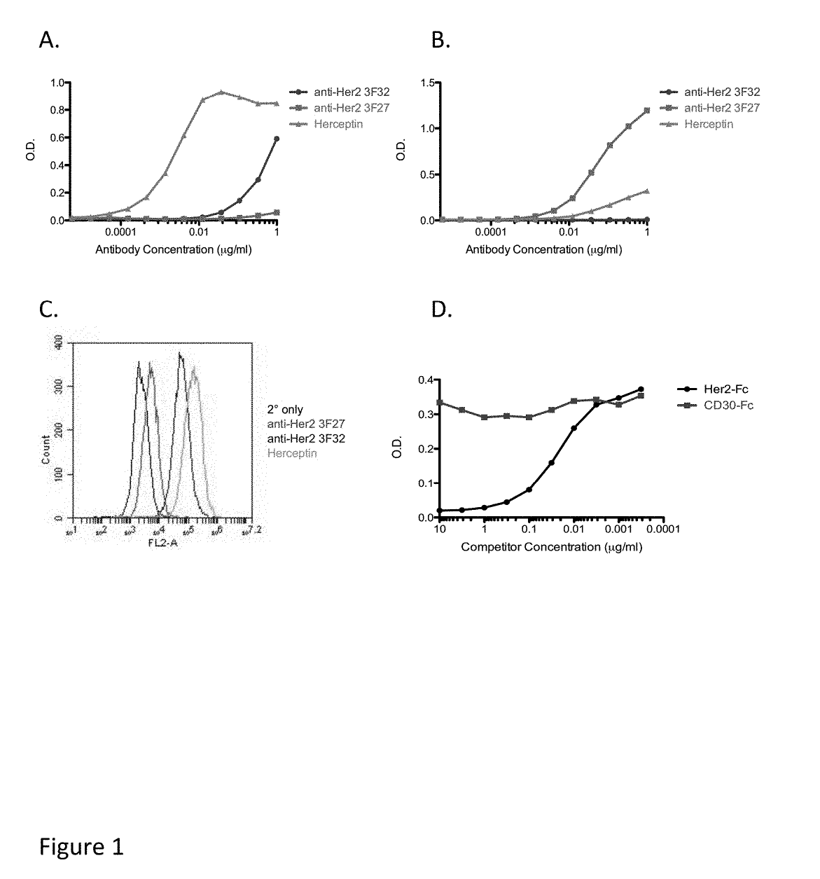

To demonstrate that the generated MAPcL-rFc proteins were designed to be folded into a native conformation, an ELISA analysis was performed using commercially available anti-HER-2 antibodies generated against either native (monoclonal antibody 3F32 and Herceptin) or denatured (monoclonal antibody 3F27) HER-2 protein. Two antigens consisting of the ECD of HER-2 were analyzed: the conformation-carrying HER-2-ECD-rFc protein generated in 293T cells and a His-HER-2-ECD protein that was produced in bacteria and purified over a nickel column. The anti-native HER-2 antibody (3F32) recognized the HER-2-ECD-rFc produced in 293T (FIG. 1A), but was unable to detect the purified His-HER-2-ECD protein produced in bacteria (FIG. 1B). Also, Herceptin was unable to detect the denatured His-HER-2-ECD protein purified from bacteria (FIG. 1B). However, a strong response was observed for Herceptin when HER-2-ECD-rFc protein was used as the antigen for the ELISA analysis (FIG. 1A). Although the 3F27 antibody generated against denatured HER-2 did not detect the HER-2-ECD-rFc protein (FIG. 1A), this antibody had a strong response to bacterial HER-2-ECD (FIG. 1B).

To confirm the specific recognition of native versus denatured epitopes by the purchased antibodies, flow cytometry was performed on unfixed SKBR3 cells, a BCa cell line known to have HER-2 amplification (38). Because surface HER-2 would retain its native confirmation on the unfixed SKBR3 cells, the anti-HER-2 3F27 antibody, specific for denatured HER-2, was unable to detect surface HER-2 on the cell membrane of SKBR3 cells by flow cytometry (FIG. 1C). When anti-HER-2 3F32 antibody and Herceptin, both of which recognize conformational HER-2, were used for flow cytometry analysis, a large shift in fluorescence was observed indicated that the antibodies recognized HER-2 present on the membrane of the SKBR3 cells (FIG. 1C).

A binding competition assay was performed to verify that the conformation-carrying antigen ELISA was recognizing the MAPcL antigen specifically. Wells were precoated with anti-rabbit IgG followed by HER-2-ECD-rFc. Purchased HER-2-Fc and CD30-Fc purified chimeric proteins (R&D Systems) were serially diluted and added to a constant amount of Herceptin (10 ng/ml) in each well. Following the addition of the HRP-conjugated secondary anti-human IgG antibody, the reactions were developed. Herceptin binding to HER-2-ECD-rFc was competed by addition of HER-2-Fc but not the CD30-Fc protein (FIG. 1D). This result indicates that Herceptin is binding specifically to the HER-2-ECD portion of the conformation-carrying fusion protein.

### Screening of Patients for Autoantibodies Using the Conformation-Carrying Antigen ELISA

Twenty MAPcL-rFc fusion antigens designed to contain their native conformation were generated by cloning the sequences encoding the ECD or secreted proteins 5′ of the rFc sequence (see Table 1 for identity of all 20 antigens). The expression plasmids were individually transfected into 293T cells, and the MAPcL-rFc fusion proteins were secreted into the media. The 20 fusion proteins were quantitated by sandwich ELISA analysis (data not shown). To detect autoantibodies in plasma collected from patients, a conformation-carrying antigen ELISA was developed using the generated MAPcL-rFc antigens. To immobilize the MAPcL-rFc fusion proteins, anti-rabbit IgG was used to precoat the wells of a 96-well plate. The media from the transfected 293T cells, which contains the generated MAPcL-rFc fusion proteins designed to have native conformations, was added to the precoated wells. To reduce plate variation and increase repeatability of the assay, three replicate samples using the plasma from each individual patient were distributed across the 96-well plate. After addition of an HRP-conjugated secondary anti-human IgG antibody, the plates were developed and the absorbance of each well was measured. The 200 plasma samples collected from newly diagnosed BCa patients and plasma from 200 age-matched healthy subjects were evaluated for autoantibody reactivity against the 20 antigens using the conformation-carrying ELISA.

The 200 BCa patients and 200 healthy controls had a mean (SD) age of 59 (11) years and 97% self identified as white race (Table 2). Cancer patients were more overweight (29.7 vs. 27.1 kg/m², p<0.0001) and had different smoking habits (p=0.014), such that there was a greater prevalence of current smokers (11% vs. 4%) in the cancer subjects versus healthy. The 200 BCa patients represented the heterogeneity of the disease consisting of 74% invasive, 24% lymph node involvement, 86% ER-positive, 17% HER-2 positive and 12% triple negative BCa (Table 2). Analyzing the absorbance reading of the autoantibody responses against the individual antigens, we determined that there were significant Bonferroni adjusted differences between BCa patients and controls in autoantibody responses against 12 TAAs, i.e. ANGPTL4, DKK1, EPHA2, GAL1, HER-2, IGFBP2, LAMC2, MUC1, SPON2, CST2, SPINT2 and SSR2 (Table 3). Higher levels of these autoantibodies were detected in BCa patients. In logistic regression models adjusted for age, race, BMI and current smoking status, autoantibody responses against MUC1 (1.83), DKK1 (1.77) and GAL1 (1.75) (all p<0.0001) had the largest odds ratios (OR), such that a patient was about 1.8 times as likely to have BCa per 1 SD increase in autoantibody response against any of these three antigens (Table 3). Autoantibody responses against six of the twelve antigens (i.e. GAL1, DKK1, MUC1, ANGPTL4, EPHA2 and IGFBP2) also increased the area under the ROC curve when each of them was added individually to the base logistic regression model adjusted for age, BMI, race and current smoking status (all p<0.05). Five of the six models were well calibrated across probability deciles (minimum Hosmer-Lemeshow p=0.13), but the model including IGFBP2 was not calibrated (p=0.016).

TABLE 3
Absorbance Measurements of Autoantibodies and their Association with Breast Cancer

| | Normal | | | | | | |
|---|---|---|---|---|---|---|---|
| | Mammogram | Breast Cancer | | Odds | | Increase in | |
| Autoantibody | (n = 200) | (n = 200) | P-value* | Ratio† | 95% CI | c-statistic‡ | P-value |
| CD320 | 0.15 (0.12) | 0.16 (0.12) | 0.62 | 1.10 | 0.90-1.35 | 0.000 | 0.96 |
| EPHA2 | 0.13 (0.06) | 0.16 (0.10) | 0.0006 | 1.64 | 1.21-2.24 | 0.034 | 0.037 |
| GFRA1 | 0.18 (0.06) | 0.20 (0.08) | 0.0081 | 1.28 | 1.03-1.59 | 0.013 | 0.32 |
| IGFBP2 | 0.21 (0.12) | 0.25 (0.13) | 0.0006 | 1.39 | 1.10-1.75 | 0.030 | 0.050 |
| CST2 | 0.17 (0.09) | 0.20 (0.10) | 0.0013 | 1.39 | 1.12-1.73 | 0.026 | 0.13 |
| GAL1 | 0.17 (0.06) | 0.20 (0.07) | <0.0001 | 1.75 | 1.37-2.23 | 0.051 | 0.021 |
| HER-2 | 0.13 (0.04) | 0.15 (0.06) | <0.0001 | 1.65 | 1.28-2.13 | 0.039 | 0.054 |
| LAMC2 | 0.15 (0.05) | 0.17 (0.08) | 0.0007 | 1.47 | 1.16-1.88 | 0.025 | 0.13 |
| ANGPTL4 | 0.18 (0.05) | 0.20 (0.06) | 0.0001 | 1.57 | 1.24-1.99 | 0.041 | 0.032 |
| DKK1 | 0.18 (0.10) | 0.24 (0.11) | <0.0001 | 1.77 | 1.40-2.24 | 0.060 | 0.0093 |
| MUC1 | 0.14 (0.06) | 0.18 (0.08) | <0.0001 | 1.83 | 1.41-2.37 | 0.055 | 0.012 |
| SSR2 | 0.14 (0.07) | 0.17 (0.08) | 0.0007 | 1.53 | 1.23-1.92 | 0.029 | 0.14 |
| LRP10 | 0.14 (0.05) | 0.15 (0.07) | 0.0098 | 1.35 | 1.09-1.68 | 0.011 | 0.47 |
| LRRC15 | 0.11 (0.04) | 0.12 (0.05) | 0.30 | 1.09 | 0.89-1.34 | 0.001 | 0.82 |
| SPINT2 | 0.15 (0.07) | 0.18 (0.09) | 0.0022 | 1.40 | 1.13-1.74 | 0.018 | 0.31 |
| SPON2 | 0.14 (0.07) | 0.17 (0.08) | <0.0001 | 1.65 | 1.31-2.07 | 0.042 | 0.052 |
| CD147 | 0.10 (0.05) | 0.12 (0.06) | 0.0039 | 1.43 | 1.15-1.78 | 0.016 | 0.38 |
| CDH3 | 0.10 (0.04) | 0.12 (0.04) | 0.0033 | 1.43 | 1.14-1.79 | 0.014 | 0.40 |
| GRN | 0.12 (0.06) | 0.13 (0.07) | 0.19 | 1.16 | 0.94-1.43 | 0.004 | 0.65 |
| SUSD2 | 0.12 (0.04) | 0.13 (0.05) | 0.0085 | 1.36 | 1.10-1.70 | 0.013 | 0.38 |

Data shown as mean (SD) of \sqrt{O.D. − Background};
*Differences between groups were tested using t-tests;
Significant Bonferroni adjusted p-value <0.05/20 = 0.0025 are shown in bold;
†Odds ratio (95% CI) for breast cancer prevalence per 1 SD increase in autoantibody was determined using logistic regression models adjusted for age, race, BMI and current smoking status;
‡Change in area under the ROC curve (i.e. c-statistic) was determined when autoantibody was added to the adjusted logistic regression models.

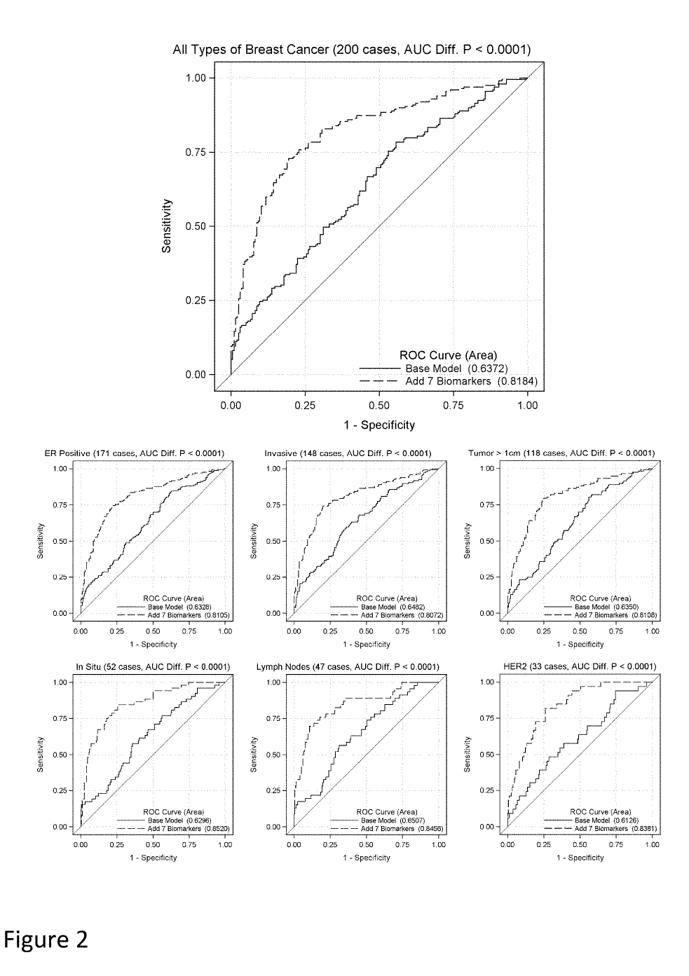

To increase the predictive ability of the conformation-carrying ELISA, the autoantibody response against a group of antigens was determined using conditional logistic regression analysis incorporating the individual age-matching study design and adjusting for BMI, race and current smoking status. The group with the best model fit (i.e. minimum AIC) contained the autoantibody responses against the following 7 antigens: ANGPTL4, DKK1, GAL1, MUC1, GFRA1, GRN and LRRC15 (Table 4). Of these 7, only autoantibody responses against ANGPTL4, DKK1, MUC1 and GAL1 individually showed a significant increase in the area under the ROC curve when added to the base model (Table 3). In the fully adjusted logistic regression model including the group of antigens, current smoking had the largest OR (95% CI) of prevalent BCa OR=7.88 (2.68-23.2); and BMI was also a significant risk factor OR=1.09 (1.04-1.13) per 1 kg/m² increase (Table 4). GAL1 had an OR of 6.73 (3.42-13.3), so a patient was almost 7 times as likely to have BCa per 1 SD increase in autoantibody response against GAL1. The autoantibody responses against GFRA1 (OR=0.41), GRN (OR=0.55) and LRRC15 (OR=0.32) all had inverse associations with odds of prevalent BCa when adjusted for responses against the other antigens (Table 4). Taken together, the autoantibody response against the group of 7 antigens increased the area under the ROC curve from 0.64 to 0.82 (p<0.0001) and had the following diagnostic measures: sensitivity (72.9%), specificity (76.0%), and positive likelihood ratio (95% CI) 3.04 (2.34 to 3.94) (FIG. 2). The model was also calibrated across risk deciles (Hosmer-Lemeshow, p=0.13).

TABLE 4
Multivariable Logistic Regression
Model Odds Ratios for Breast Cancer

| Variable | Odds Ratio | 95% CI | |
|---|---|---|---|
| Age (per 1 year) | 1.00* | 0.98 | 1.02 |
| White Race | 0.70 | 0.19 | 2.68 |
| BMI (per 1 kg/m²) | 1.09 | 1.04 | 1.13 |
| Current Smoking | 7.88 | 2.68 | 23.2 |
| ANGPTL4 (per 1 SD) | 1.71 | 1.16 | 2.50 |
| DKK1 (per 1 SD) | 1.87 | 1.28 | 2.73 |
| GAL1 (per 1 SD) | 6.73 | 3.42 | 13.3 |
| GFRA1 (per 1 SD) | 0.41 | 0.21 | 0.82 |
| GRN (per 1 SD) | 0.55 | 0.38 | 0.81 |
| LRRC15 (per 1 SD) | 0.32 | 0.19 | 0.55 |
| MUC1 (per 1 SD) | 1.67 | 1.16 | 2.41 |

*Due to individual 1:1 age-matching.

Because BCa is a heterogeneous disease, it is possible that the autoantibody response against a combination of antigens may categorize a subtype of BCa differently than analyzing all BCa subtypes as a whole. The BCa samples were grouped into individual BCa subtypes: invasive, in situ, ER positive, tumor maximum dimension >1 cm, lymph node involvement and HER-2 positive. The ability to discriminate cases from controls in each subtype was tested using autoantibody reactivity against the 7-antigen combination in addition to age, BMI, race and current smoking status (FIG. 2). The 7-antigen combination model performed similarly in all subtypes of BCa; the c-statistic was 0.81 to 0.85. Of the BCa subtypes, in situ tumors had the greatest area under the ROC curve (0.8520, p<0.0001) when analyzed for autoantibody responses against the 7-antigen combination. The model was not calibrated when considering only those cancers with lymph node involvement due to four unexpected BCas with very low model probabilities (Hosmer-Lemeshow p=0.0036).

### DISCUSSION

Early detection of BCa allows a physician to treat the initial stage of the disease before metastasis, thereby allowing for a higher rate of remission or long-term survival for the patient. Detecting the presence of autoantibodies generated against tumor proteins in the blood of patients would be an ideal method for BCa detection. However, the tumor antigens need to be identified before specific autoantibody responses in patients can be ascertained. We generated a library that encodes membrane and secreted proteins that are highly expressed in BCa and may elicit an immune response.

We have shown that antigen conformation alters antibody-binding affinity in our assay, and the detection of autoantibodies is limited by epitope conformation (FIG. 1). We used a robust sample set to develop the conformation-carrying ELISA consisting of 200 plasma samples collected from newly diagnosed BCa patients before surgery, chemotherapy or radiation treatment. In addition, plasma was collected from 200 age-matched subjects defined by a confirmed normal mammogram in the preceding six months (Table 2). All 400 plasma samples were screened individually for autoantibody response against 20 TAAs designed to contain their native conformation using ELISA. Four of the 20 TAAs analyzed in our assay have previously been reported to generate an antibody response in BCa patients: MUC1 (39, 40), HER-2 (41), IGFBP2 (15) and GRN (42). Detection of autoantibodies against 12 of the 20 antigens was statistically significant for discriminating between normal and cancer samples (Table 3, bold). However, we did not observe a significant autoantibody response against GRN in our assay. Of the 12 significant antigens, 9 have not been previously associated with BCa autoantibodies. To our knowledge, this is the first report of the detection of autoantibodies against ANGPTL4, CST2, DKK1, EPHA2, GAL1, LAMC2, SPINT2, SPON2 and SSR2 in BCa patients (Table 3).

Previously it has been shown that screening serum against a panel of antigens to detect autoantibodies compared to only a single antigen increases the sensitivity of the assay (17). This finding is consistent with the fact that BCa is a heterogeneous disease (43), and each individual patient's immune system is distinct. A combination of seven TAAs, consisting of ANGPTL4, DKK1, GAL1, MUC1, GFRA1, GRN and LRRC15, had the greatest diagnostic capability (Table 4). Compared to previously published multiple antigen panels used to detect BCa autoantibodies (17, 44-46), the combination of these seven TAAs is unique, and our study contains the largest patient population of BCa and healthy samples. Interestingly, in the seven-antigen combination, four of the antigens have statistical significance individually (Table 3), but three of the antigens, GFRA1, GRN and LRRC15, were not statistically significant on their own (Table 3). However, GFRA1, GRN and LRRC15 were inversely associated with BCa, indicating that lower amounts of these autoantibodies in a patient, in combination with higher levels of the directly associated autoantibodies, increased the likelihood of having BCa (Table 4). When the 7 antigens were added to knowledge of current smoking status and BMI, the sensitivity and specificity of the assay was 72.9% and 76.0%, respectively. The area under the ROC curve (95% CI) was 0.82 (0.77 to 0.85), and the positive likelihood ratio was 3.04 for the conformation-carrying ELISA. Because BCa is a heterogeneous disease, patients were grouped into tumor characteristics, including ER positive, HER-2 positive, in situ, invasive, tumor size and lymph node involvement. The 7-antigen combination performed well for all groups (FIG. 2). These results suggest that the assay has potential clinical application. One serum recurrence marker for BCa that is currently used in the clinic is mucin-associated antigen CA27.29. The CA27.29 antigen is detected in the blood of a patient using a monoclonal antibody that recognizes MUC1. Because of the low sensitivity of the CA27.29 tumor marker, the test is used to follow a patient for BCa recurrence (47). Compared to the traditional CA27.29 tumor marker, the conformation-carrying ELISA described here shows great promise.

Currently, mammography is the standard method for BCa screening. However, the machinery necessary to perform a mammogram is expensive, requires specialized medical personnel to operate and is challenging to transport to medically underserved areas. The development of a blood test for the early detection of BCa would greatly advance access to screening. Drawing blood is a common procedure, and blood can easily be mailed to a clinical laboratory for analysis. This study demonstrates that a combination of autoantibody responses against antigens designed to contain conformational epitopes is a promising strategy for BCa detection. Future studies will focus on the identification of additional antigens to improve the sensitivity and specificity of the assay for translation into the clinic.

TABLE 5
Autoantibody combination subsets and their association with breast cancer

| | | | | | ROC | Increase in |
|---|---|---|---|---|---|---|
| Plex | Sets of Autoantibodies | Sensitivity % | Specificity % | PLR | AUC | ROC AUC* |
| 7 | ANGPTL4 DKK1 GAL1 GFRA1 | 72.9 | 76.0 | 3.04 | 0.818 | 0.181 |
| | GRANULIN LRRC15 MUC1 | | | | | |
| 6 | ANGPTL4 DKK1 GAL1 GRANULIN | 72.4 | 75.5 | 2.96 | 0.810 | 0.173 |
| | LRRC15 MUC1 | | | | | |
| 4 | ANGPTL4 DKK1 GAL1 LRRC15 | 69.8 | 74.5 | 2.74 | 0.790 | 0.152 |
| 5 | ANGPTL4 DKK1 GAL1 GFRA1 | 69.3 | 74.5 | 2.72 | 0.803 | 0.165 |
| | LRRC15 | | | | | |
| 6 | DKK1 GAL1 GFRA1 GRANULIN | 70.4 | 74.0 | 2.71 | 0.809 | 0.172 |
| | LRRC15 MUC1 | | | | | |
| 6 | ANGPTL4 DKK1 GAL1 GFRA1 | 70.9 | 73.5 | 2.68 | 0.812 | 0.175 |
| | GRANULIN LRRC15 | | | | | |
| 5 | DKK1 GAL1 GRANULIN LRRC15 | 69.8 | 73.0 | 2.59 | 0.805 | 0.167 |
| | MUC1 | | | | | |
| 5 | DKK1 GAL1 GFRA1 GRANULIN | 68.3 | 73.5 | 2.58 | 0.799 | 0.162 |
| | LRRC15 | | | | | |
| 5 | DKK1 GAL1 GFRA1 LRRC15 MUC1 | 68.8 | 73.0 | 2.55 | 0.797 | 0.160 |
| 5 | ANGPTL4 DKK1 GAL1 GRANULIN | 68.3 | 73.0 | 2.53 | 0.804 | 0.166 |
| | LRRC15 | | | | | |
| 4 | DKK1 GAL1 GFRA1 LRRC15 | 68.3 | 73.0 | 2.53 | 0.791 | 0.153 |
| 4 | DKK1 GAL1 GRANULIN LRRC15 | 68.8 | 72.4 | 2.49 | 0.796 | 0.159 |
| 5 | ANGPTL4 DKK1 GAL1 LRRC15 | 69.8 | 71.4 | 2.44 | 0.794 | 0.157 |
| | MUC1 | | | | | |
| 3 | DKK1 GAL1 LRRC15 | 66.8 | 72.4 | 2.42 | 0.784 | 0.147 |
| 4 | ANGPTL4 GAL1 LRRC15 MUC1 | 66.8 | 72.4 | 2.42 | 0.784 | 0.147 |
| 4 | GAL1 GFRA1 LRRC15 MUC1 | 68.3 | 71.4 | 2.39 | 0.788 | 0.151 |
| 3 | GAL1 GFRA1 LRRC15 | 66.8 | 71.9 | 2.38 | 0.770 | 0.132 |
| 3 | ANGPTL4 GAL1 LRRC15 | 71.6 | 69.8 | 2.37 | 0.774 | 0.137 |
| 4 | DKK1 GAL1 LRRC15 MUC1 | 67.8 | 71.4 | 2.37 | 0.789 | 0.152 |
| 4 | ANGPTL4 GAL1 GFRA1 LRRC15 | 68.8 | 70.9 | 2.36 | 0.793 | 0.156 |
| 3 | GAL1 LRRC15 MUC1 | 67.3 | 71.4 | 2.35 | 0.778 | 0.141 |
| 5 | ANGPTL4 GAL1 GFRA1 LRRC15 | 68.8 | 69.9 | 2.29 | 0.798 | 0.161 |
| | MUC1 | | | | | |
| 3 | ANGPTL4 GAL1 GFRA1 | 64.8 | 66.3 | 1.92 | 0.753 | 0.116 |
| 3 | DKK1 GAL1 GFRA1 | 65.3 | 65.8 | 1.91 | 0.746 | 0.109 |
| 3 | GAL1 GFRA1 MUC1 | 63.3 | 64.8 | 1.80 | 0.746 | 0.109 |

PLR = positive likelihood ratio, sensitivity/(100 − specificity);
ROC = receiver operating characteristic curve;
AUC = area under the curve;
*Change in area under the ROC curve (i.e. c-statistic) was determined when the set of autoantibodies was added to a logistic regression model adjusted for age, race, BMI, and current smoking status (all p-value <0.0001).

### REFERENCES

1. National Cancer Institute at the National Institutes of Health 2012 [updated Jul. 24, 2012; cited 2013]. Available from: http://www.cancer.gov/cancertopics/factsheet/detection/mammograms.
2. Breastcancer.org, Mammography: Benefits, Risks, What You Need to Know 2013. Available from: http://www.breastcancer.org/symptoms/testing/types/mammograms/benefits_risks.jsp.
3. American Cancer Society, Find Support & Treatment, Mammograms and Other Breast Imaging Procedures 2012. Available from: http://www.cancer.org/Treatment/UnderstandingYour Diagnosis/ExamsandTestDescriptions/MammogramsandOtherBreastImagingProcedures/mammo grams-and-other-breast-imaging-procedures-having-a-mammogram.
4. Agnantis N J, Goussia A C, Stefanou D. Tumor markers. An update approach for their prognostic significance. Part I. In Vivo. 2003; 17(6):609-18. PubMed PMID: 14758728.
5. Arciero C, Somiari S B, Shriver C D, Brzeski H, Jordan R, Hu H, et al. Functional relationship and gene ontology classification of breast cancer biomarkers. Int J Biol Markers. 2003; 18(4):241-72. PubMed PMID: 14756541.
6. Bernoux A, de Cremoux P, Laine-Bidron C, Martin E C, Asselain B, Magdelenat H. Estrogen receptor negative and progesterone receptor positive primary breast cancer: pathological characteristics and clinical outcome. Institut Curie Breast Cancer Study Group. Breast Cancer Res Treat. 1998; 49(3):219-25. PubMed PMID: 9776505.
7. Dowsett M, Cooke T, Ellis I, Gullick W J, Gusterson B, Mallon E, et al. Assessment of HER2 status in breast cancer: why, when and how? Eur J Cancer. 2000; 36(2):170-6. PubMed PMID: 10741274.
8. Shak S. Overview of the trastuzumab (Herceptin) anti-HER2 monoclonal antibody clinical program in HER2-overexpressing metastatic breast cancer. Herceptin Multinational Investigator Study Group. Semin Oncol. 1999; 26(4 Suppl 12):71-7. PubMed PMID: 10482196.
9. Slamon D J, Clark G M, Wong S G, Levin W J, Ullrich A, McGuire W L. Human breast cancer: correlation of relapse and survival with amplification of the HER-2/neu oncogene. Science. 1987; 235(4785):177-82. PubMed PMID: 3798106.
10. Slamon D J, Godolphin W, Jones L A, Holt J A, Wong S G, Keith D E, et al. Studies of the HER-2/neu proto-oncogene in human breast and ovarian cancer. Science. 1989; 244(4905):707-12. PubMed PMID: 2470152.
11. Kaklamani V. A genetic signature can predict prognosis and response to therapy in breast cancer: Oncotype DX. Expert review of molecular diagnostics. 2006; 6(6):803-9. Epub 2006/12/05. doi: 10.1586/14737159.6.6.803. PubMed PMID: 17140367.
12. Manjili M H, Najarian K, Wang X Y. Signatures of tumor-immune interactions as biomarkers for breast cancer prognosis. Future Oncol. 2012; 8(6):703-11. Epub 2012/07/07. doi: 10.2217/fon.12.57. PubMed PMID: 22764768.
13. Reuschenbach M, von Knebel Doeberitz M, Wentzensen N. A systematic review of humoral immune responses against tumor antigens. Cancer immunology, immunotherapy: CII. 2009; 58(10):1535-44. Epub 2009/06/30. doi: 10.1007/s00262-009-0733-4. PubMed PMID: 19562338; PubMed Central PMCID: PMC2782676.
14. Casiano C A, Mediavilla-Varela M, Tan E M. Tumor-associated antigen arrays for the serological diagnosis of cancer. Mol Cell Proteomics. 2006; 5(10):1745-59. Epub 2006/05/31. doi: R600010-MCP200 [pii] 10.1074/mcp.R600010-MCP200. PubMed PMID: 16733262.
15. Lu H, Goodell V, Disis M L. Humoral Immunity Directed against Tumor-Associated Antigens As Potential Biomarkers for the Early Diagnosis of Cancer. J Proteome Res. 2008; 7(4):1388-94. PubMed PMID: 18311901.
16. Desmetz C, Mange A, Maudelonde T, Solassol J. Autoantibody signatures: progress and perspectives for early cancer detection. Journal of cellular and molecular medicine. 2011; 15(10):2013-24. Epub 2011/06/10. doi: 10.1111/j.1582-4934.2011.01355.x. PubMed PMID: 21651719.
17. Piura E, Piura B. Autoantibodies to tailor-made panels of tumor-associated antigens in breast carcinoma. Journal of oncology. 2011; 2011:982425. Epub 2011/03/23. doi: 10.1155/2011/982425. PubMed PMID: 21423545; PubMed Central PMCID: PMC3056218.
18. Piura E, Piura B. Autoantibodies to tumor-associated antigens in breast carcinoma. Journal of oncology. 2010; 2010:264926. Epub 2010/11/30. doi: 10.1155/2010/264926. PubMed PMID: 21113302; PubMed Central PMCID: PMC2989457.
19. Finn O J. Immune response as a biomarker for cancer detection and a lot more. N Engl J Med. 2005; 353(12):1288-90. PubMed PMID: 16177255.
20. Pavoni E, Pucci A, Vaccaro P, Monteriu G, Ceratti Ade P, Lugini A, et al. A study of the humoral immune response of breast cancer patients to a panel of human tumor antigens identified by phage display. Cancer Detect Prev. 2006; 30(3):248-56. PubMed PMID: 16876336.
21. Sioud M, Hansen M H. Profiling the immune response in patients with breast cancer by phage-displayed cDNA libraries. Eur J Immunol. 2001; 31(3):716-25. PubMed PMID: 11241275.
22. Storr S J, Chakrabarti J, Barnes A, Murray A, Chapman C J, Robertson J F. Use of autoantibodies in breast cancer screening and diagnosis. Expert Rev Anticancer Ther. 2006; 6(8):1215-23. PubMed PMID: 16925487.
23. Tan E M, Shi F D. Relative paradigms between autoantibodies in lupus and autoantibodies in cancer. Clin Exp Immunol. 2003; 134(2):169-77. PubMed PMID: 14616773.
24. Barlow D J, Edwards M S, Thornton J M. Continuous and discontinuous protein antigenic determinants. Nature. 1986; 322(6081):747-8. PubMed PMID: 2427953.
25. Laver W G, Air G M, Webster R G, Smith-Gill S J. Epitopes on protein antigens: misconceptions and realities. Cell. 1990; 61(4):553-6. PubMed PMID: 1693095.
26. Ramachandran N, Hainsworth E, Bhullar B, Eisenstein S, Rosen B, Lau A Y, et al. Self-assembling protein microarrays. Science. 2004; 305(5680):86-90. Epub 2004/07/03. doi: 10.1126/science.1097639 305/5680/86 [pii]. PubMed PMID: 15232106.
27. Ehrlich J R, Qin S, Liu B C. The ‘reverse capture’ autoantibody microarray: a native antigen-based platform for autoantibody profiling. Nature protocols. 2006; 1(1):452-60. Epub 2007/04/05. doi: 10.1038/nprot.2006.66. PubMed PMID: 17406268.
28. Tan H T, Low J, Lim S G, Chung M C. Serum autoantibodies as biomarkers for early cancer detection. The FEBS journal. 2009; 276(23):6880-904. Epub 2009/10/29. doi: 10.1111/j.1742-4658.2009.07396.x. PubMed PMID: 19860826.
29. Egland K A, Vincent J J, Strausberg R, Lee B, Pastan I. Discovery of the breast cancer gene BASE using a molecular approach to enrich for genes encoding membrane and secreted proteins. Proc Natl Acad Sci USA. 2003; 100(3):1099-104. PubMed PMID: 12538848.
30. Boyle J S, Koniaras C, Lew A M. Influence of cellular location of expressed antigen on the efficacy of DNA vaccination: cytotoxic T lymphocyte and antibody responses are suboptimal when antigen is cytoplasmic after intramuscular DNA immunization. Int Immunol. 1997; 9(12):1897-906. PubMed PMID: 9466317.
31. Drew D R, Lightowlers M, Strugnell R A. Humoral immune responses to DNA vaccines expressing secreted, membrane bound and non-secreted forms of the Tania ovis 45W antigen. Vaccine. 2000; 18(23):2522-32. PubMed PMID: 10775786.
32. Bendtsen J D, Nielsen H, von Heijne G, Brunak S. Improved prediction of signal peptides: SignalP 3.0. J Mol Biol. 2004; 340(4):783-95. doi: 10.1016/j jmb.2004.05.028. PubMed PMID: 15223320.
33. Nielsen H, Engelbrecht J, Brunak S, von Heijne G. Identification of prokaryotic and eukaryotic signal peptides and prediction of their cleavage sites. Protein engineering. 1997; 10(1):1-6. PubMed PMID: 9051728.
34. Krogh A, Larsson B, von Heijne G, Sonnhammer E L. Predicting transmembrane protein topology with a hidden Markov model: application to complete genomes. J Mol Biol. 2001; 305(3):567-80. doi: 10.1006/jmbi.2000.4315. PubMed PMID: 11152613.
35. Bergstralh E J, Kosanke J L. Computerized matching of cases to controls. Rochester, Minn.: Mayo Clinic Department of Health Science Research, 1995.
36. Hosmer D W, Lemeshow S. Goodness of fit tests for the multiple logistic regression model. Communications in Statistics—Theory and Methods: Taylor & Francis Group; 1980. p. 1043-69.
37. Akaike H. Information Theory and an Extension of the Maximum Likelihood Principle. In: Kotz S, Johnson N L, editors. Breakthroughs in Statistics. Springer Series in Statistics: Springer New York; 1992. p. 610-24.
38. Neve R M, Chin K, Fridlyand J, Yeh J, Baehner F L, Fevr T, et al. A collection of breast cancer cell lines for the study of functionally distinct cancer subtypes. Cancer Cell. 2006; 10(6):515-27. Epub 2006/12/13. doi: 10.1016/j.ccr.2006.10.008. PubMed PMID: 17157791; PubMed Central PMCID: PMC2730521.
39. Kotera Y, Fontenot J D, Pecher G, Metzgar R S, Finn O J. Humoral immunity against a tandem repeat epitope of human mucin MUC-1 in sera from breast, pancreatic, and colon cancer patients. Cancer Res. 1994; 54(11):2856-60. Epub 1994/06/01. PubMed PMID: 7514493.
40. von Mensdorff-Pouilly S, Gourevitch M M, Kenemans P, Verstraeten A A, Litvinov S V, van Kamp G J, et al. Humoral immune response to polymorphic epithelial mucin (MUC-1) in patients with benign and malignant breast tumours. Eur J Cancer. 1996; 32A(8):1325-31. Epub 1996/07/01. PubMed PMID: 8869094.
41. Disis M L, Pupa S M, Gralow J R, Dittadi R, Menard S, Cheever M A. High-titer HER-2/neu protein-specific antibody can be detected in patients with early-stage breast cancer. J Clin Oncol. 1997; 15(11):3363-7. Epub 1997/11/18. PubMed PMID: 9363867.
42. Ladd J J, Chao T, Johnson M M, Qiu J, Chin A, Israel R, et al. Autoantibody signatures involving glycolysis and splicesome proteins precede a diagnosis of breast cancer among postmenopausal women. Cancer Res. 2013; 73(5):1502-13. Epub 2012/12/28. doi: 10.1158/0008-5472.CAN-12-2560. PubMed PMID: 23269276.
43. Perou C M, Sorlie T, Eisen M B, van de Rijn M, Jeffrey S S, Rees C A, et al. Molecular portraits of human breast tumours. Nature. 2000; 406(6797):747-52. Epub 2000/08/30. doi: 10.1038/35021093. PubMed PMID: 10963602.
44. Lacombe J, Mange A, Jarlier M, Bascoul-Mollevi C, Rouanet P, Lamy P J, et al. Identification and validation of new autoantibodies for the diagnosis of DCIS and node negative early-stage breast cancers. Int J Cancer. 2013; 132(5):1105-13. Epub 2012/08/14. doi: 10.1002/ijc.27766. PubMed PMID: 22886747.
45. Mange A, Lacombe J, Bascoul-Mollevi C, Jarlier M, Lamy P J, Rouanet P, et al. Serum autoantibody signature of ductal carcinoma in situ progression to invasive breast cancer. Clin Cancer Res. 2012; 18(7):1992-2000. Epub 2012/02/11. doi: 10.1158/1078-0432.CCR-11-2527. PubMed PMID: 22322670.
46. Ye H, Sun C, Ren P, Dai L, Peng B, Wang K, et al. Mini-array of multiple tumor-associated antigens (TAAs) in the immunodiagnosis of breast cancer. Oncology letters. 2013; 5(2):663-8. Epub 2013/02/20. doi: 10.3892/01.2012.1062. PubMed PMID: 23420714; PubMed Central PMCID: PMC3573153.
47. Gion M, Mione R, Leon A E, Dittadi R. Comparison of the diagnostic accuracy of CA27.29 and CA15.3 in primary breast cancer. Clin Chem. 1999; 45(5):630-7. Epub 1999/05/01. PubMed PMID: 10222349.

We claim:

1. A method for detecting breast cancer or disease recurrence, comprising
(a) contacting a bodily fluid sample from a subject at risk of having breast cancer or breast cancer recurrence with one or more proteins selected from the group consisting of ANGTPL4, DKK1, EPHA2, LAMC2, SPON2, SSR2, GAL1, GFRA1, LRRC15, CD147, CD320, CDH3, LRP10, SPINT2, SUSD2, and CST2, secreted versions thereof, or extracellular domains thereof; and
(b) detecting binding of autoantibodies in the bodily fluid sample against the one or more proteins, secreted versions thereof, or extracellular domains thereof;
wherein the presence of autoantibodies against the one or more proteins, secreted versions thereof, or extracellular domains thereof correlates with a likelihood of the subject having breast cancer or breast cancer recurrence.

2. The method of claim 1, wherein the one or more proteins comprise one or more of ANGPTL4, DKK1, EPHA2, GAL1, LAMC2, SPON2, CST2, SPINT2 and SSR2, secreted versions thereof, or extracellular domains thereof.

3. The method of claim 1, wherein the contacting step comprises contacting the bodily fluid sample with two or more proteins selected from the group consisting of ANGTPL4, DKK1, EPHA2, LAMC2, SPON2, SSR2, GAL1, GFRA1, LRRC15, CD147, CD320, CDH3, LRP10, SPINT2, SUSD2, and CST2, secreted versions thereof, or extracellular domains thereof.

4. The method of claim 1, wherein the contacting step comprises contacting the bodily fluid sample with five or more proteins selected from the group consisting of ANGTPL4, DKK1, EPHA2, LAMC2, SPON2, SSR2, GAL1, GFRA1, LRRC15, CD147, CD320, CDH3, LRP10, SPINT2, SUSD2, and CST2, secreted versions thereof, or extracellular domains thereof.

5. The method of claim 1, wherein the contacting step comprises contacting the bodily fluid sample with proteins in of the following marker sets, secreted versions thereof, or extracellular domains thereof:
ANGPTL4, DKK1, GAL1, GFRA1, GRANULIN, LRRC15, and MUC1;
ANGPTL4, DKK1, GAL1, GRANULIN, LRRC15, and MUC1;
ANGPTL4, DKK1, GAL1, and LRRC15;
ANGPTL4, DKK1, GAL1, GFRA1, and LRRC15;
DKK1, GAL1, GFRA1, GRANULIN, LRRC1, and 5 MUC1;
ANGPTL4, DKK1, GAL1, GFRA1, GRANULIN, and LRRC15;
DKK1, GAL1, GRANULIN, LRRC15, and MUC1;
DKK1, GAL1, GFRA1, GRANULIN, and LRRC15;
DKK1, GAL1, GFRA1, LRRC15, and MUC1;
ANGPTL4, DKK1, GAL1, GRANULIN, and LRRC15;
DKK1, GAL1, GFRA1, and LRRC15;
DKK1, GAL1, GRANULIN, and LRRC15;
ANGPTL4, DKK1, GAL1, LRRC15, and MUC1;
DKK1, GAL1, and LRRC15;
ANGPTL4, GAL1, LRRC15, and MUC1;
GAL1, GFRA1, LRRC15, and MUC1;
GAL1, GFRA1, and LRRC15;
ANGPTL4, GAL1, and LRRC15;
DKK1, GAL1, LRRC15, and MUC1;
ANGPTL4, GAL1, GFRA1, and LRRC15;
GAL1, LRRC15, and MUC1;
ANGPTL4, GAL1, GFRA1, LRRC15, and MUC1;
ANGPTL4, GAL1, and GFRA1;
DKK1, GAL1, and GFRA1; and
GAL1, GFRA1, and MUC1.

6. The method of claim 1, wherein the contacting step comprises contacting the bodily fluid sample ANGPTL4, DKK1, GAL1, MUC1, GFRA1, GRN and LRRC15, secreted versions thereof, or extracellular domains thereof.

7. The method of claim 1, wherein the bodily fluid sample comprises a serum sample from the subject.

8. The method of claim 1, wherein the bodily fluid sample comprises a blood sample from the subject.

9. The method of claim 1, wherein the contacting comprises use of ELISA.

10. The method of claim 1, wherein the contacting comprises use of Longitudinal Assay Screening, wherein all target biomarkers are detected and quantitated within a single test and dilution.

11. The method of claim 1, wherein the one or more proteins, secreted versions thereof, or extracellular domains thereof are detectably labeled.

12. The method of claim 1, wherein the one or more proteins, secreted versions thereof, or extracellular domains thereof are immobilized on a surface.

13. The method of claim 1, wherein the method identifies the subject as likely to have breast cancer or breast cancer recurrence, and wherein the method further comprises treating the subject with an amount of a therapeutic sufficient to treat the breast cancer or breast cancer recurrence.
